SEQUENCE LISTING (4 sequences)

SEQ ID NO: 1 - DNA, 1267 bp, Homo sapiens
cgagggctgcttccggctggtgcccccgggggagacccaacctggggcgacttcaggggtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactcaaaaaaaaaaaaaa

SEQ ID NO: 2 - DNA, 422 bp, Homo sapiens
ccccggtcgcgctttctctgccctccgcccgggtggacctggagcgcttgagcggtcggcgcgcctggagcagccaggcgggcagtggactagctgctggaccagggaggtgtgggagagcggtggcggcgggtacatgcacgtgaagccattgcgagaactttatccataagtatttcaatgccggtagggacggcaagagaggagggcgggatgtgccacacatctttgacctcaggtttctaacgcctgttttctttctgccctctgcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaa

SEQ ID NO: 3 - DNA, 616 bp, Artificial Sequence
  Probe
gtgctgtcactccctttaccagtgggtctgcattttcctttgcactacaagtactctaactacagacattgttccggtttttatctgattataagtatccaactaaagtagttaaatgtttagaaaaggtaaataactgatagtgcataagagaacctgaagtactctgttgacacttaaattatatagccaaggacaatggaggaaatcatttagtgctattaatcattaatctaacagtattgtgtctgtgtgcaactgtctttcttttggctaatatctgtgaattaattggtcattgaaaaatctgataaaggaatattctaaaagtacacagtgaaatccaaattatttctatcaattacagctcaccttttctgaggcttaaacttgacatgtaaaaacattttagttatctgtttcaaaatgaacaatatacctctggttaaaattctaccttttaaaaattaaggtatttgagacctacagggattaattctctacttaatatttactttactttagtagtttttctacagaaagtttgtgaaagatttcatcccctatatatgtttctttatcctccaaaatctggtcctaccactgtactccttga

SEQ ID NO: 4 - DNA, 854 bp, Artificial Sequence
  Probe
cctgagtataagcaaagcgctgtccgtggtgcccctttaaagggaatgcccccggcaacgttttccctacccaaagcttttattcattcagtcaacttgcttcgcgaagctcacacatctgcctcgtgcaagattctcagtcattttacttagtcattggttctttccctatcaccattctttatgtccccctcaaagaaaaacattatcttccatttccttatcaactccaaacagctttcatttttctgacatatttactacctaagaaaatggctcaagaattgggtcagactatcttgtcctaacttttctgataagtttcagagaaactcaaaggtcaaaacaagagcataagagtaaaaggtagagttttttttaaactgaagactaggaaatgggggttggatgggaaagaaaaagaaattgttattaatgctacccggttcccttccctgtccaggtggatttcagctctgttgaggctctgtcagtagattttcagccctaaccagcacttccatggtggtggcacttccactgcctttaaaagaaagagctttttttaattctacagggatttgggggatgaggagtcagagctaaggtatcctaaaaaaaacatgtgaagactctcattttgcaatacacaagcaattgccctcctgttaagactttgtcttcctcagcactccgaaccaaaatgattctgtaaacaaaaattgttcacttttaggagaggtccacttatgcagttcctcacccaagtttttaggcaacaaatccataacttgcggttctcttcctatccaatgtagcatccgctgaaatgttttaaatattttaagtaataa

(19) United States
(12) Patent Application Publication
(10) Pub. No.: US 2016/0153052 A1
(43) Pub. Date: Jun. 2, 2016

(54) MARKER TO PREDICT AND MONITOR RESPONSE TO AURORA KINASE B INHIBITOR THERAPY

(71) Applicant: AbbVie Inc., North Chicago, IL (US)

(72) Inventors: Daniel Albert, Highland Park, IL (US); Peter Ansell, Grayslake, IL (US); Paul Hessler, Hainesville, IL (US); Lloyd Lam, Libertyville, IL (US); Chris Tse, Libertyville, IL (US)

(73) Assignee: AbbVie Inc., North Chicago, IL (US)

(21) Appl. No.: 14/957,059

(22) Filed: Dec. 2, 2015

Related U.S. Application Data
(60) Provisional application No. 62/086,330, filed on Dec. 2, 2014.

(51) Int. Cl. C12Q 1/68

(52) U.S. Cl. CPC C12Q 1/6886; C12Q 2600/106; C12Q 2600/158

(57) ABSTRACT

The present invention relates to diagnostic assays useful in classification of patients for selection of cancer therapy with one or more Aurora kinase B inhibitors. In particular, the present invention relates to identifying the genetic defects in the CDKN2A locus which exhibit a heightened dependency on Aurora kinase activity, identifying patients eligible to receive Aurora kinase inhibitor therapy, either as monotherapy or as part of combination therapy, and monitoring patient response to such therapy.

### RELATED APPLICATION INFORMATION

This application claims the benefit of U.S. Provision Application No. 62/086,330, filed Dec. 2, 2014, which is herein incorporated by reference in its entirety.

### FIELD OF THE INVENTION

The present invention relates to diagnostic assays useful in classification of patients for selection of cancer therapy with one or more Aurora kinase B inhibitors. In particular, the present invention relates to identifying the genetic defects in the CDKN2A locus which exhibit a heightened dependency on Aurora kinase activity, identifying patients eligible to receive Aurora kinase inhibitor therapy, either as monotherapy or as part of combination therapy, and monitoring patient response to such therapy.

### BACKGROUND

The Aurora kinase family is a group of highly related serine/threonine kinases that function as key regulators of mitosis. Three Aurora kinases are expressed in mammalian cells. These Aurora kinases are Aurora A, Aurora B, and Aurora C. Each of these Aurora kinases exhibits a different subcellular localization and plays a distinct role (See, Carmena M. E. W., Nat. Rev. Mol. Cell Biol., 4:842-854 (2003) and (Ducat, D. Z. Y., Exp. Cell Res., 301:60-67 (2004)). Specifically, Aurora A localizes to spindle poles and has a crucial role in bipolar spindle formation (See, Marumoto, T. Z. D., et al., Nat. Rev. Cancer, 5:42-50 (2005)). Aurora B, a chromosome passenger protein, localizes to centromeres in early mitosis and then the spindle midzone in anaphase. Aurora B is required for mitotic histone H3 phosphorylation, chromosome biorientation, the spindle assembly checkpoint and cytokinesis (Andrews, P. D., et al., Curr. Opin. Cell Biol., 15:672-683 (2003)). Aurora C is also a chromosomal passenger protein and, in normal cells, its expression is restricted to the testis where it functions primarily in male gametogenesis. As the Aurora kinases serve essential functions in mitosis, considerable attention has been given to targeting this family of kinases for cancer therapy. Several small-molecule inhibitors have been developed including Hesperadin, ZM447439, VX-680/MK0457, AZD1152, MLN8054 (See, Ditchfield, C, J. V., et al., J. Cell Biol., 161:267-280 (2003), Harrington, E. A., et al., Nat. Med., 10:262-267 (2004), Hauf, S., et al., J. Cell Biol., 161:281-294 (2003), Manfredi, M. G., et al., Proc. Natl. Acad. Sci., USA, 104:4106-4111 (2007)), and 1-(4-(4-amino-7-(1-(2-hydroxyethyl)-1H-pyrazol-4-yl)thieno[3,2-c]pyridin-3-yl)phenyl)-3-(3-fluorophenyl)urea (ABT-348) (See, Glaser, K. B., et al., J. Pharmacol Exp. Ther., 353(3):617-627 (2012)).

Genetic heterogeneity of cancer is a factor complicating the development of efficacious cancer drugs. Cancers that are considered to be a single disease entity according to classical histopathological classification often reveal multiple genomic subtypes when subjected to molecular profiling. In certain cases, different genomic subtypes appear to have functional relevance to the efficacy of certain drugs. For example, the efficacy of certain targeted cancer drugs has been correlated with the presence of a genomic feature, such as gene amplification or gene mutations. For the 2^{nd} generation anti-mitotic agents being investigated (e.g., Aurora kinase inhibitors, Polo kinase inhibitors, Cyclin-dependent kinase inhibitors), there exists a need for the identification of patients most likely to respond to such therapies. Previous methods relied on clinical trials with large patient enrollment strategies with the hope of identifying patients whose genomics subtypes are sensitive to the agent(s). However, this approach is time consuming, expensive, often not effective, and may unnecessarily subject patients to ineffective therapy. The present invention overcomes the issue of patient selection for Aurora B kinase inhibitors by selecting patients whose cancers exhibit genetic ablation of the cyclin-dependent kinase 2A (CDKN2A) locus.

### SUMMARY

In its first aspect, the present invention relates to methods of classifying a patient for eligibility for treatment with an Aurora kinase inhibitor, the method comprising the steps of: a) providing a test sample from a patient; b) determining the presence or absence of a copy number decrease of the cyclin dependent kinase 2A (CDKN2A) locus in the sample; and c) classifying the patient as being eligible for receiving treatment with an Aurora kinase inhibitor if the copy number decrease of the CDKN2A locus is present.

In some embodiments, the CDKN2A locus comprises SEQ ID NO:1 or a sequence substantially identical to SEQ ID NO:1.

In some embodiments, the CDKN2A locus comprises a p16 gene.

In some embodiments, the p16 gene comprises SEQ ID NO:2 or a sequence substantially identical to SEQ ID NO:2.

In some embodiments, the determining step (b) is performed by in situ hybridization.

In some embodiments, the determining step (b) is performed with at least one probe comprising a sequence complementary to CDKN2A or SEQ ID NO:1.

In some embodiments, the determine step (b) is performed with at least one probe comprising a sequence complementary to p16 or SEQ ID NO:2.

In some embodiments, the probe comprises at least one sequence selected from group consisting of the sequences SEQ ID NO:3 and SEQ ID NO:4.

In some embodiments, the probe is fluorescently labeled.

In some embodiments, the determining step (b) is performed by polymerase chain reaction.

In some embodiments, the determining step (b) is performed by a nucleic acid microarray assay.

In some embodiments, the Aurora kinase inhibitor is selected from the group consisting of Aurora kinase A inhibitors, Aurora kinase B inhibitors, Aurora kinase C inhibitors, and mixtures thereof. In a specific embodiment, the Aurora kinase inhibitor is 1-(4-(4-amino-7-(1-(2-hydroxyethyl)-1H-pyrazol-4-yl)thieno[3,2-c]pyridin-3-yl)phenyl)-3-(3-fluorophenyl)urea.

In some embodiments, the patient is also being treated with chemotherapy, radiation or combinations thereof.

In some embodiments, the test sample comprises a tissue sample.

In its second aspect, the present invention relates to methods of monitoring a patient suffering from cancer and being treated with an Aurora kinase inhibitor, the method comprising the steps of: a) providing a test sample from a patient suffering from cancer and currently being treated with at least one Aurora kinase inhibitor; b) determining a copy number for the cyclin dependent kinase 2A (CDKN2A) locus in the sample; c) comparing the copy number of the CDKN2A locus in the sample against a predetermined level; and d) determining that the patient should continue to be treated with the Aurora kinase inhibitor if the copy number of the CDKN2A locus in the sample is lower than the predetermined level.

In its third aspect, the present invention relates to kits for classifying a patient for eligibility for treatment with an Aurora kinase inhibitor comprising at least one reagent capable of detecting the presence or absence of a copy number decrease of the cyclin dependent kinase 2A (CDKN2A) locus in the sample, wherein the presence of the copy number decrease of the CDKN2A locus indicates the patient as being eligible for receiving treatment with an Aurora kinase inhibitor.

In some embodiments, the CDKN2A comprises a p16 gene.

In some embodiments, the reagent comprises at least one probe comprising a sequence complementary to the CDKN2A.

In some embodiments, the reagent comprises at least one probe comprising a sequence complementary to the p16 gene.

In some embodiments, the reagent comprises at least one antibody immunologically specified for the proteins encoded by CDKN2A.

In some embodiments, the antibody is used in an ELISA.

In its fourth aspect, the present invention relates to kits for monitoring a patient suffering from cancer and being treated with an Aurora kinase inhibitor comprising at least one reagent capable of detecting the presence or absence of a copy number decrease of the cyclin dependent kinase 2A (CDKN2A) locus in the sample, wherein the presence of the copy number decrease of the CDKN2A locus indicates that the patient should continue to be treated with the Aurora kinase inhibitor.

### BRIEF DESCRIPTION OF THE DRAWINGS

The patent or application file contains at least one drawing executed in color. Copies of this patent or patent application with colored drawings will be provided by the Office upon request and payment of the necessary fee.

FIG. 1 shows CDKN2A deletion associated with clinical response in patients with solid tumors treated with ABT-348 in clinical trial.

FIG. 2 shows CDKN2A status of Objective Responders in clinical trial.

FIG. 3 shows percentage of cycling cells at baseline associated with pharmacodynamic activity of patients treated with ABT-348.

FIG. 4 shows the identification of CDKN2A as a sensitive biomarker for ABT-348.

FIG. 5 shows relative potencies of other Aurora kinase inhibitors.

FIG. 6 shows that the predictive nature of CDKN2A extends to multiple Aurora kinase inhibitors.

FIG. 7 shows that silencing p16 sensitizes OCI-AML2 cells to Aurora kinase B inhibitor compounds ABT-348 and AZD-1152, but not ABT-869.

FIG. 8 shows the approaches that were employed to test CDKN2A status clinically.

### DETAILED DESCRIPTION

### Embodiments

The present invention is based on, at least in part, the discovery that the expression levels of genes in cyclin dependent kinase 2A locus are associated with the therapeutic responses of patients who receive Aurora kinase inhibitor treatment.

In first aspect, the present invention provides methods of classifying a patient for eligibility for treatment with an Aurora kinase inhibitor, the method comprising the steps of: a) providing a test sample from a patient; b) determining the presence or absence of a copy number decrease of the cyclin dependent kinase 2A (CDKN2A) locus in the sample; and c) classifying the patient as being eligible for receiving treatment with an Aurora kinase inhibitor if the copy number decrease of the CDKN2A locus is present.

In some embodiments, the CDKN2A locus comprises SEQ ID NO:1 or a sequence substantially identical to SEQ ID NO:1.

In some embodiments, the CDKN2A comprises a p16 gene.

In some embodiments, the p16 gene comprises SEQ ID NO:2 or a sequence substantially identical to SEQ ID NO:2.

In some embodiments, the determining step (b) is performed by in situ hybridization. Preferably, the in situ hybridization is performed with at least two nucleic acid probes. More preferably, the nucleic acid probes are fluorescently labeled.

In some embodiments, the probe comprises a sequence complementary to CDKN2A or SEQ ID NO:1.

In some embodiments, the probe comprises a sequence complementary to p16 or SEQ ID NO:2.

In some specification embodiments, the probe comprises at least one sequence selected from group consisting of the sequences of SEQ ID NO:3 and SEQ ID NO:4.

In some embodiments, the determining step (b) is performed by polymerase chain reaction.

In some embodiments, the determining step (b) is performed by a nucleic acid microarray assay.

In some embodiments, the Aurora kinase inhibitor is selected from the group consisting of Aurora kinase A inhibitors, Aurora kinase B inhibitors, Aurora kinase C inhibitors, and mixtures thereof. Preferably, the Aurora kinase inhibitor is an Aurora kinase B inhibitor selected from a group consisting of AZD1152, ZM447439, VX-680/MK0457, 1-(4-(4-amino-7-(1-(2-hydroxyethyl)-1H-pyrazol-4-yl)thieno[3,2-c]pyridin-3-yl)phenyl)-3-(3-fluorophenyl)urea (ABT-348), and Hersperadin. More preferably, the Aurora kinase B inhibitor is ABT-348.

In some embodiments, the test sample comprises a tissue sample. For example, the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

In some embodiments, the treatment is for cancer. Preferably, the treatment is for the cancer selected from the group consisting of acoustic neuroma, acute leukemia, acute lymphoblastic leukemia, acute myelogenous leukemia, acute t-cell leukemia, basal cell carcinoma, bile duct carcinoma, bladder cancer, brain cancer, breast cancer, bronchogenic carcinoma, Burkitt's lymphoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, dysplasias, metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer, lymphangioendothelio-sarcoma, lymphangiosarcoma, lymphoblastic leukemia, lymphoma, malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors, stomach cancer, squamous cell carcinoma, synovioma, sweat gland carcinoma, testicular cancer, thyroid cancer, Waldenstrom's macroglobulinemia, testicular tumors, uterine cancer, and Wilms' tumor. In another embodiment of the methods herein, the treatment is for cancer, and the cancer is acute myeloid leukemia.

In some embodiments, the patient is also being treated with chemotherapy, radiation or combinations thereof. In another embodiment of the methods herein, the patient is also being treated with chemotherapy. In another embodiment of the methods herein, the patient is also being treated with radiation. In another embodiment of the methods herein, the patient is also being treated with chemotherapy and radiation.

In its second aspect, the present invention provides methods of monitoring a patient suffering from cancer and being treated with an Aurora kinase inhibitor, the method comprising the steps of: a) providing a test sample from a patient suffering from cancer and currently being treated with at least one Aurora kinase inhibitor; b) determining a copy number for the cyclin dependent kinase 2A (CDKN2A) locus in the sample; c) comparing the copy number of the CDKN2A locus in the sample against a predetermined level; and d) determining that the patient should continue to be treated with the Aurora kinase inhibitor if the copy number of the CDKN2A locus in the sample is lower than the predetermined level.

In its third aspect, the present invention provides a kit for classifying a patient for eligibility for treatment with an Aurora kinase inhibitor comprising at least one reagent capable of detecting the presence or absence of a copy number decreases or increases of the cyclin dependent kinase 2A (CDKN2A) locus in the sample, wherein the presence of the copy number decrease of the CDKN2A locus indicate the patient as being eligible for receiving treatment with an Aurora kinase inhibitor.

Generally, the reagent can be any molecules that are capable of binding the genes of CDKN2A or p16, or the gene products thereof and providing detectable measurement. The examples of reagents include, but are not limited to chemical compounds including organic or inorganic compounds, antibodies, single or double stranded oligonucleotides, amino acids, proteins, peptides or fragments thereof.

In some embodiments, the reagent comprises at least one probe comprising a sequence complementary to the CDKN2A.

In some embodiments, the reagent comprises at least one probe comprising a sequence complementary to the p16 gene.

In some embodiments, the reagent comprises at least one probe comprising a sequence selected from a group consisting of SEQ ID NO:3 and SEQ ID NO:4.

In some embodiments, the reagent comprises antibodies immunologically specified for the proteins encoded by CDKN2A.

In some embodiments, the antibodies are used in an ELISA.

In its fourth aspect, the present invention provides a kit for monitoring a patient suffering from cancer and being treated with an Aurora kinase inhibitor comprising at least one reagent capable of detecting the presence or absence of a copy number decreases or increases of the cyclin dependent kinase 2A (CDKN2A) locus in the sample, wherein the presence of the copy number decrease indicates that the patient should continue to be treated with the Aurora kinase inhibitor.

### Description of the Biomarkers

As disclosed herein, biomarkers for the purpose of this invention are derived from cyclin dependent kinase 2A.

The term “CDKN2A”, as described herein, refers to the human cyclin-dependent kinase inhibitor 2A gene on chromosome 9p21. The gene is sometimes also called “CDKN2”, “CDK4 inhibitor”, “”MTS1″, “TP16”, “p16 (INK4)”, “p16 (INK4A)”, “p14(ARF)”, “p12”, or “p16-gamma”. The sequence information of the CDKN2A of the present invention is available at NCBI under Reference Sequence No. NM_000077.4.

In some embodiments, the CDKN2A biomarker of the present invention comprises a sequence of SEQ ID NO:1 or contiguous portions thereof, or a sequence substantially identical to SEQ ID NO:1. Preferably, the CDKN2A comprises a sequence at least 90%, at least 95%, at least 98%, or at least 99% identical to SEQ ID NO:1 or the contiguous portions thereof.

TABLE 1
CDKN2A cDNA NCBI Reference Sequence: NM_000077.4
(SEQ ID NO: 1)

| | |
|---|---|
| 1 | cgagggctgc ttccggctgg tgcccccggg ggagacccaa cctggggcga cttcaggggt |
| 61 | gccacattcg ctaagtgctc ggagttaata gcacctcctc cgagcactcg ctcacggcgt |
| 121 | ccccttgcct ggaaagatac cgcggtccct ccagaggatt tgagggacag ggtcggaggg |
| 181 | ggctcttccg ccagcaccgg aggaagaaag aggaggggct ggctggtcac cagagggtgg |
| 241 | ggcggaccgc gtgcgctcgg cggctgcgga gagggggaga gcaggcagcg ggcggcgggg |
| 301 | agcagcatgg agccggcggc ggggagcagc atggagcctt cggctgactg gctggccacg |
| 361 | gccgcggccc ggggtcgggt agaggaggtg cgggcgctgc tggaggcggg ggcgctgccc |
| 421 | aacgcaccga atagttacgg tcggaggccg atccaggtca tgatgatggg cagcgcccga |
| 481 | gtggcggagc tgctgctgct ccacggcgcg gagcccaact gcgccgaccc cgccactctc |
| 541 | acccgacccg tgcacgacgc tgcccgggag ggcttcctgg acacgctggt ggtgctgcac |
| 601 | cgggccgggg cgcggctgga cgtgcgcgat gcctggggcc gtctgcccgt ggacctggct |
| 661 | gaggagctgg gccatcgcga tgtcgcacgg tacctgcgcg cggctgcggg gggcaccaga |
| 721 | ggcagtaacc atgcccgcat agatgccgcg gaaggtccct cagacatccc cgattgaaag |
| 781 | aaccagagag gctctgagaa acctcgggaa acttagatca tcagtcaccg aaggtcctac |
| 841 | agggccacaa ctgcccccgc cacaacccac cccgctttcg tagttttcat ttagaaaata |
| 901 | gagcttttaa aaatgtcctg ccttttaacg tagatatatg ccttccccca ctaccgtaaa |
| 961 | tgtccattta tatcattttt tatatattct tataaaaatg taaaaaagaa aaacaccgct |
| 1021 | tctgcctttt cactgtgttg gagttttctg gagtgagcac tcacgcccta agcgcacatt |
| 1081 | catgtgggca tttcttgcga gcctcgcagc ctccggaagc tgtcgacttc atgacaagca |
| 1141 | ttttgtgaac tagggaagct caggggggtt actggcttct cttgagtcac actgctagca |
| 1201 | aatggcagaa ccaaagctca aataaaaata aaataatttt cattcattca ctcaaaaaaa |
| 1261 | aaaaaaa |

The CDKN2A of the present invention may also refer to a sequence comprises a p16 gene. The sequence information of the p16 gene is available at GeneBank under Accession number AH005371.2.

In some embodiments, the CDKN2A of the present invention comprises a p16 gene comprising a sequence of SEQ ID NO:2 or contiguous portions thereof, or a sequence substantially identical to SEQ ID NO:2. Preferably, the p16 comprises a sequence at least 90%, at least 95%, at least 98%, or at least 99% identical SEQ ID NO:2 or the contiguous portions thereof.

TABLE 2
Homo sapiens p16-INK4 (p16) gene, complete cds
(SEQ ID NO: 2)

| | |
|---|---|
| 1 | ccccggtcgc gctttctctg ccctccgccc gggtggacct ggagcgcttg agcggtcggc |
| 61 | gcgcctggag cagccaggcg ggcagtggac tagctgctgg accagggagg tgtgggagag |
| 121 | cggtggcggc gggtacatgc acgtgaagcc attgcgagaa ctttatccat aagtatttca |
| 181 | atgccggtag ggacggcaag agaggagggc gggatgtgcc acacatcttt gacctcaggt |
| 241 | ttctaacgcc tgttttcttt ctgccctctg cagacatccc cgattgaaag aaccagagag |
| 301 | gctctgagaa acctcgggaa acttagatca tcagtcaccg aaggtcctac agggccacaa |
| 361 | ctgcccccgc cacaacccac cccgctttcg tagttttcat ttagaaaata gagcttttaa |
| 421 | aa |

The term “sequence identity” as used herein refers to the extent that sequences are identical on a nucleotide-by-nucleotide basis or an amino acid-by-amino acid basis over a window of comparison. Nucleic acid and protein sequence identities can be evaluated by using any method known in the art. For example, the identities can be evaluated by using the Basic Local Alignment Search Tool (“BLAST”). The BLAST programs identity homologous sequences by identifying similar segments between a query amino or nucleic acid sequence and a test sequence which is preferably obtained from protein or nuclei acid sequence database. The BLAST program can be used with the default parameters or with modified parameters provided by the user.

The “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, GIy, VaI, Leu, lie, Phe, Tyr, Trp, Lys, Arg, His, Asp, GIu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like.

The term “substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%.

### Definitions

Section headings as used in this section and the entire disclosure herein are not intended to be limiting.

Throughout this application, the term “about” is used to indicate that a value includes the inherent variation of error for the device, the method being employed to determine the value, or the variation that exists among the study subjects. When used in the context of % reduction of activity, concentration, number of DNA bases, number of cells, number of nucleotides, temperature, time, amount (including weight, volume and equivalents), or number of carbon atoms, the term “about” is used to indicate a value of ±10% from the reported value, preferably a value of ±5% from the reported value.

Biomarkers

The term “biomarker” or “marker” refers to an organic biomolecule which is differentially present in a sample taken from a subject of one phenotypic status as compared with another phenotypic status. A biomarker is differentially present between different phenotypic statuses if the difference in the mean or median expression levels of the biomarker in the different groups is calculated to be statistically significant. For the purpose of this invention, biomarkers are the markers for classifying patients eligible for receiving Aurora kinase inhibitor treatment or monitoring patients who have received Aurora kinase inhibitors to determine whether the treatment of Aurora kinase inhibitors should be continued in those patients. One skilled in the art would understand that in addition to a gene itself, a biomarker also includes the product of that gene.

Gene and Locus

The term “gene” used herein refers to a nucleic acid fragment that expresses a specific protein, including regulatory sequences preceding (5′ non-coding sequences) and following (3′ non-coding sequences) the coding sequence.

The term “locus” used herein refers the specific location of a particular DNA sequence on a chromosome. In some embodiments, a particular DNA sequence can be of any length (e.g., one, two, three, ten, fifty, or more nucleotides). In some embodiments, the locus is or comprises a gene or a portion of a gene. In some embodiments, the locus is or comprises an exon or a portion of an exon of a gene. In some embodiments, the locus is or comprises an intron or a portion of an intron of a gene. In some embodiments, the locus is or comprises a regulatory element or a portion of a regulatory element of a gene. In some embodiments, the locus is associated with a disease, disorder, and/or condition. For example, mutations at the locus (including deletions, insertions, splicing mutations, point mutations, etc.) may be correlated with a disease, disorder, and/or condition.

Gene Expression Level

The term “expression” refers to the production of a functional end-product. Expression of a gene involves transcription of the gene and translation of the mRNA into a precursor or mature protein.

The term “expression level” of a gene as used herein refers to the measurable quantity of gene product produced by the gene in a sample of a patient wherein the gene product can be a transcriptional product or a translated transcriptional product. Accordingly, the expression level can pertain to a nucleic acid gene product such as RNA or cDNA or a polypeptide. The expression level is derived from a subject/patient sample and/or a control sample, and can, for example, be detected de novo or correspond to a previous determination.

The expression level can also refer to a copy number of a gene in the locus. The term “copy number” or “gene copy number” as used in reference to specific nucleic acid sequences (e.g., CDKN2A) refers to the actual number of these sequences per single test sample (e.g., cell). Copy number may be reported for one single sample, or reported as the average number in a group of samples (e.g., tissue sample).

Determination or Measurement of Gene Expression

The term “determining an expression level” or “expression level is determined” as used in reference to the process of determining whether a gene is expressed and if this is the case assessing to which extend it is expressed. The process can be an application of an agent and/or method to a sample, for example a sample from the subject and/or a control sample, for ascertaining quantitatively, semi-quantitatively or qualitatively the amount of a gene expression product, for example the amount of polypeptide, RNA, DNA, gene copies present in a sample. Therefore, the process of determining gene expression may include all necessary preparatory steps known in the art such as protein, mRNA, RNA, DNA and/or cDNA preparation; measurement using techniques such as real time PCR, immunohistochemistry or microarray; basic arithmetic operations such as determining a mean value, if gene expression level for one biological sample is determined using more than one probe since the average of the probes can then be calculated in order to increase the accuracy of the inventive method; etc.

The process can be carried out by any techniques known in the art including, but are not limited to, microarray, mass spectrometry, polymerase chain reaction (PCR), reverse transcription PCR, real-time PCR, in-situ hybridization, southern dot blots, immunoassay, ribonuclease protection assay cDNA array techniques, ELISA, protein, antigen or antibody arrays on solid supports such as glass or ceramics, and small interfering RNA functional assays. Preferably, the process is performed by polymerase chain reaction (PCR). In some embodiments, the process may further comprise a nucleic acid microarray assay.

In some embodiments, the expression of a gene can also be determined or measured by the polypeptide level of the gene product through immunoassay. For example, the expression of a gene can be determined by methods including, but not limited to, Western blot, flow cytometry, immunohistochemistry, ELISA, immunoprecipation and the like.

In some preferred embodiments, the process of the present invention is to determine or measure a copy number of CDKN2A locus in a sample. Preferably, the process is to determine a copy number decrease or increase of CDKN2A locus compared to a pre-determined level.

The term “predetermined level” refers to an assay cut-off value that is used to assess diagnostic results by comparing the assay results against the predetermined level, and where the predetermined level already that has been linked or associated with various clinical parameters (e.g., assessing risk, severity of disease, progression/non-progression/improvement, determining the age of a test sample, determining whether a test sample (e.g., serum or plasma) has hemolyzed, etc.).

In some embodiments, a predetermined level is a measured level of the biomarker to a control or to one or more previous measurements, carried out at different points of time, of the level of the biomarker in the same patient.

In some embodiments, a predetermined level is a “baseline level”. The term “baseline level” refers to a control level, and in some embodiments, a normal level, of biomarker or gene expression or activity against to which the expression level of biomarker or gene can be compared. Therefore, it can be determined, based on the control or baseline level of biomarker whether a sample has a measurable increase, decrease, or substantially no change in biomarker level, as compared to the baseline level.

In one embodiment, the baseline level can be established from a previous sample from the same subject. For example, the baseline level of a biomarker is established in an autologous control sample obtained from the subject. That is, the sample is obtained from the same subject from which the sample to be evaluated is obtained. The control sample is preferably the same sample type as the sample to be evaluated.

The method for establishing a baseline level of a biomarker is preferably the same method that will be used to evaluate the sample from the subject. In a preferred embodiment, the baseline level is established using the same sample type as the sample to be evaluated.

The present invention provides exemplary predetermined levels, and describes the initial linkage or association of such levels with clinical parameters for exemplary assays as described herein. However, it is well known that cutoff values may vary dependent on the nature of the assay. It further is well within the ordinary skill of one in the art to adapt the invention herein for other assays to obtain assay-specific cut-off values for those other assays based on this description.

When comparing a copy number of gene in a test sample to a pre-determined level, one may not need to determine the exact copy number of the sample, but instead would obtain an approximation that allows one to determine whether a given sample contains more or less of the nucleic acid sequence as compared to another sample. Thus, any method capable of reliably directly or indirectly determining amounts of nucleic acid may be used as a measure of copy number even if the actual copy number is not determined.

The term “decrease” or “reduction” means the observed difference of a gene expression in a test sample less than a predetermined level or a baseline level. The observed difference can be carried out by any means known in the art, for example, either through measurement or analysis of a test sample.

In some embodiments, a decrease is at least 2-5%, 5-10%, 10-15%, 10-20%, 20-30%, 30-40%, 40-50%, 50-60%, 60-70%, 70-80%, 80-90% or 90-100% less than a predetermined level. In some embodiments, a decrease is at least 10%, 20%, 30%, 40%, 50%, 60%, 70%, 80%, 90%, 100% or above, less than a predetermined level.

In some embodiments, a decrease means the expression level of CDKN2A measured or analyzed in a test sample is lower relative to a pre-determined level. Non-limiting examples include, at least 2-5%, 5-10%, 10-15%, 10-20%, 20-30%, 30-40%, 40-50%, 50-60%, 60-70%, 70-80%, 80-90% and 90-100% less than a predetermined level of CDKN2A expression. In some embodiments, a decrease is at least 10%, 20%, 30%, 40%, 50%, 60%, 70%, 80%, 90%, 100% or above, less than a predetermined level of CDKN2A expression.

Of course, one skilled in the art would understand that for the purpose of classifying a patient according to the present invention, a copy number decrease of a gene may vary over a number factors, such as different diseases, indications of the patients, as well as characteristics of the patients including, but not limited to, age, gender, physiology, disease state, activity level, or activity profile.

The term “increase” means the observed difference of a gene expression in a test sample greater than a predetermined level. The observed difference can be carried out by any means known in the art, for example, either through measurement or analysis of a test sample.

In some embodiments, an increase is at least 2-5%, 5-10%, 10-15%, 10-20%, 20-30%, 30-40%, 40-50%, 50-60%, 60-70%, 70-80%, 80-90% and 90-100% greater than a predetermined level. In some embodiments, a increase is at least 10%, 20%, 30%, 40%, 50%, 60%, 70%, 80%, 90%, 100% or above, greater than a predetermined level.

In some embodiments, an increase means the expression level of CDKN2A measured or analyzed in a test sample is higher relative to a pre-determined level. Non-limiting examples, include but are not limited to, at least 2-5%, 5-10%, 10-15%, 10-20%, 20-30%, 30-40%, 40-50%, 50-60%, 60-70%, 70-80%, 80-90% and 90-100% higher than a predetermined level of CDKN2A expression. In some embodiments, an increase is at least 10%, 20%, 30%, 40%, 50%, 60%, 70%, 80%, 90%, 100% or above, higher than a predetermined level of CDKN2A expression.

Of course, one skilled in the art would understand that for the purpose of classifying a patient according to the present invention, a copy number increase of a gene may vary over a number of factors such as different diseases, indications of the patients, as well as characteristics of the patients, including, but not limited to, age, gender, physiology, disease state, activity level, or activity profile.

Polymerase Chain Reaction or PCR

As described above, the determination or measurement of the gene of the present invention is preferably performed by “Polymerase Chain Reaction” or “PCR”. PRC is a technique for the synthesis of large quantities of specific DNA segments, consisting of a series of repetitive cycles. Typically, the double stranded DNA is heat-denatured, and the two primers complementary to the 3′ boundaries of the target segment are annealed at low temperature and then extended at an intermediate temperature. One set of these three consecutive steps is referred to as a cycle.

PCR is a powerful technique used to amplify DNA millions of fold, by repeated replication of a template, in a short period of time. (Mullis, K., et al., Cold Spring Harb Symp Quant Biol. 51 Pt 1:263-73 (1986)); European Patent Application No. 50,424; European Patent Application No. 84,796; European Patent Application No. 258,017, European Patent Application No. 237,362; European Patent Application No. 201,184, U.S. Pat. No. 4,683,202; U.S. Pat. No. 4,582,788; and U.S. Pat. No. 4,683,194). The process uses sets of specific in vitro synthesized oligonucleotides to prime DNA synthesis. The design of the primers is dependent upon the sequences of DNA that are to be analyzed. The technique is carried out through many cycles (usually 20-50) of melting the template at high temperature, allowing the primers to anneal to complementary sequences within the template and then replicating the template with DNA polymerase.

The products of PCR reactions can be analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleic bases, sugars and covalent inter-nucleoside (backbone) linkages as well as polymers having non-naturally-occurring portions that function similarly. Such modified or substituted nucleic acid polymers are well known in the art and are referred to as “analogues.” Oligonucleotides are generally short polynucleotides from about 10 to up to about 160 or 200 nucleotides.

Polynucleotides also comprise primers that specifically hybridize to target sequences, including analogues and/or derivatives of the nucleic acid sequences, and homologues thereof.

Polynucleotides can be prepared by conventional techniques, such as solid-phase synthesis using commercially available equipment, such as that available from Applied Biosystems USA Inc. (Foster City, Calif.; USA), DuPont, (Wilmington, Del.; USA), or Milligen (Bedford, Mass.; USA). Modified polynucleotides, such as phosphorothioates and alkylated derivatives, can also be readily prepared by similar methods known in the art (See, U.S. Pat. Nos. 4,948,882, 5,464,746, and 5,424,414).

The term “polynucleotide analogues” refers to polymers having modified backbones or non-natural inter-nucleoside linkages. Modified backbones include those retaining a phosphorus atom in the backbone, such as phosphorothioates, chiral phosphorothioates, phosphorodithioates, phosphotriesters, aminoalkylphosphotriesters, methyl and other alkyl phosphonates, as well as those no longer having a phosphorus atom, such as backbones formed by short chain alkyl or cycloalkyl inter-nucleoside linkages, mixed heteroatom and alkyl or cycloalkyl inter-nucleoside linkages, or one or more short chain heteroatomic or heterocyclic inter-nucleoside linkages. Modified nucleic acid polymers (analogues) can contain one or more modified sugar moieties.

Analogs that are RNA or DNA mimetics, in which both the sugar and the inter-nucleoside linkage of the nucleotide units are replaced with novel groups, are also useful. In these mimetics, the base units are maintained for hybridization with the target sequence. An example of such a mimetic, which has been shown to have excellent hybridization properties, is a peptide nucleic acid (PNA) (See, Buchardt, 0., P. Nielsen, and R. Berg. 1992. Peptide Nucleic Acids).

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decrease DNA duplex stability. A common approach is to vary the temperature: higher relative temperatures result in more stringent reaction conditions (See, Ausubel, F.M., R. Brent, R. E. Kingston, et al. 1987. Current Protocols in Molecular Biology. John Wiley & Sons, New York which provides an excellent explanation of stringency of hybridization reactions).

Hybridization under “stringent conditions” means hybridization protocols in which nucleotide sequences at least 60% homologous to each other remain hybridized.

Polynucleotides can include other appended groups such as peptides (e.g., for targeting host cell receptors in vivo), or agents facilitating transport across the cell membrane. In addition, oligonucleotides can be modified with hybridization-triggered cleavage agents (See, van der Krol et al., Biotechniques. 6:958-76 (1988) or intercalculating agents (Zon, G., Pharm Res. 5:539-49 (1988)). The oligonucleotide can be conjugated to another molecule, e.g., a peptide, a hybridization triggered cross-linking agent, a transport agent, a hybridization-triggered cleavage agent, and the like.

“Target sequence” or “target nucleic acid sequence” means a nucleic acid sequence encompassing, for example, a gene, or complements or fragments thereof, that is amplified, detected, or both using a polynucleotide primer or probe. Additionally, while the term target sequence sometimes refers to a double stranded nucleic acid sequence; a target sequence can also be single-stranded. In cases where the target is double-stranded, polynucleotide primer sequences preferably amplify both strands of the target sequence. A target sequence can be selected that is more or less specific for a particular organism. For example, the target sequence can be specific to an entire genus, to more than one genus, to a species or subspecies, serogroup, auxotype, serotype, strain, isolate or other subset of organisms.

Probes and Primers

As described above, the expression of a gene of the invention can be determined or measured by hybridization using a probe or primer. The gene of the present invention can also be the basis for designing a complimentary probe or primer, to detect the presence and/or quantity of the gene expression in a subject or a test sample.

The term “probe” as used herein refers to a nucleic acid molecule that comprises a sequence of nucleotides that will hybridize specifically to a target nucleic acid sequence e.g. a coding sequence of a gene, such as CDKN2A gene disclosed herein. The probe comprises at least 10 or more, 15 or more, 20 or more bases or nucleotides that are complementary and hybridize contiguous bases and/or nucleotides in the target nucleic acid sequence. The length of probe depends on the hybridization conditions and the sequences of the probe and nucleic acid target sequence and can for example be 10-20, 21-70, 71-100, 101-500 or more bases or nucleotides in length.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH).

The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides or any number in between, although it can contain less. The factors involved in determining the appropriate length of primer are readily known to one of ordinary skill in the art.

The hybridization of nucleic acids is well understood in the art. Typically a primer can be made from any combination of nucleotides or nucleotide derivatives or analogs available in the art. The ability of such primers to specifically hybridize to polynucleotide sequences will enable them to be of use in detecting the presence of complementary sequences in a given subject. The primers of the invention can hybridize to complementary sequences in a subject such as a biological sample, including, without limitation, saliva, sputum, blood, plasma, serum, urine, feces, cerebrospinal fluid, amniotic fluid, wound exudate, or tissue of the subject. Polynucleotides from the sample can be, for example, subjected to gel electrophoresis or other size separation techniques or can be immobilized without size separation.

The probes or the primers can also be labeled for the detection. Suitable labels and methods for labeling primers are known in the art. For example, the label includes, without limitation, radioactive labels, biotin labels, fluorescent labels, chemiluminescent labels, bioluminescent labels, metal chelator labels and enzyme labels. The polynucleotides from the sample are contacted with the probes or primers under hybridization conditions of suitable stringencies. Preferably, the primer is fluorescent labeled. Also, the detection of the presence or quality of the gene sequence of interest can be accomplished by any method known in the art. For instance, the detection can be made by a DNA amplification reaction. In some embodiments, “amplification” of DNA denotes the use of polymerase chain reaction (PCR) to increase the concentration of a particular DNA sequence within mixtures of DNA sequences.

A person skilled in the art would recognize that all or part of a particular probe or primer can be used as long as the portion is sufficient for example in the case a probe, to specifically hybridize to the intended target and in the case of a primer, sufficient to prime amplification of the intended template.

Patient or Subject

As used herein, the terms “patient” and “subject” which are used interchangeably in this disclosure, refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the patient is a human. Also, subjects or patients can be living or expired.

Classification of Patients

The term “classifying” or “classification” as used herein refers to assigning, to a class or kind, an unclassified subject. A “class” or “group” then being a grouping of subjects, based on one or more characteristics, attributes, properties, qualities, effects, parameters, etc., which they have in common, for the purpose of classifying them according to an established system or scheme.

In some embodiments, patients having decreased expression of the cyclin dependent kinase 2A (CDKNA2A) are grouped or assigned to be eligible for receiving treatment with an Aurora kinase inhibitor. In contrast, patients having increased expression of the cyclin dependent kinase 2A (CDKNA2A) are grouped or assigned to be excluded from receiving treatment with an Aurora kinase inhibitor.

In other embodiments, classification can also refer to identifying a group of subjects based on a calculation of a relative level of gene expression in those subjects which would have same or similar response to a certain treatment. For example, subjects having an expression level of CDKNA2A below a pre-determined or selected threshold can be benefit from treatment with an Aurora kinase inhibitor. In contrast, a subject having an expression level of CDKNA2A equal or above a pre-determined or selected threshold are less likely to be benefit from treatment with an Aurora kinase inhibitor.

Monitoring Patients

The term “monitoring” as used herein, preferably, refers to assessing the course of a disease. Preferably, by assessing the course of the disease, it can be determined whether the condition of a subject suffering from that disease improves or deteriorates. As will be understood by those skilled in the art, such an assessment is usually not intended to be correct for 100% of the subjects to be monitored. The term, however, requires that the assessment is correct for a statistically significant portion of the subjects. Whether a portion is statistically significant can be determined without further ado by the person skilled in the art using various well known evaluation tools.

There are provided herein methods for monitoring a patient who has been treated with an Aurora kinase inhibitor, particularly in the context of determining whether the patient is responsive to the treatment and whether the treatment should be continued. The methods typically involve assessing the level of a biomarker prior to or at the time of beginning a treatment and subsequently assessing the level of the biomarker periodically throughout the treatment or on completion of the treatment.

The monitoring can also refer to assessing the development of a disease, such as the progression of the disease (i.e. worsening of the disease) or the regression of the disease (i.e. a patient's recovery).

In some embodiments of the present invention, the assessment described above can be made by comparing a gene expression level to a pre-determined level of the gene expression as defined below. In other embodiments, the assessment can also be made by comparing a gene expression level to one or more previous measurements, carried out at different points of time, of the expression level of the gene in the same patient. For example, a decreased level of the gene, compared to the result from a previous measurement or to a pre-determined level may be used to indicate the progression of the disease, while an increased level of the gene, compared to the result from a previous measurement or to a pre-determined level is used to indicate the regression of the disease.

Test Sample or Biological Sample

The term “test sample” means a sample taken from a subject, or a biological fluid, wherein the sample may contain a target sequence. A test sample can be taken from any source, for example, tissue, blood, saliva, sputa, mucus, sweat, urine, urethral swabs, cervical swabs, urogenital or anal swabs, conjunctival swabs, ocular lens fluid, cerebral spinal fluid, etc. A test sample can be used (i) directly as obtained from the source; or (ii) following a pre-treatment to modify the character of the sample. Thus, a test sample can be pre-treated prior to use by, for example, preparing plasma or serum from blood, disrupting cells or viral particles, preparing liquids from solid materials, diluting viscous fluids, filtering liquids, adding reagents, purifying nucleic acids, etc.

In some embodiments, the test sample comprises a tissue sample. In other embodiments, the test sample comprises a tissue sample, wherein the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

One skilled in the art would understand that a test sample can be prepared and/or provided by using technologies known in the art. The selection of suitable technologies for preparing and providing a test sample may vary depending on factors including, but not limited to, the type of the sample, the source of the sample, the physical or chemical properties of the sample, the determination and measurement used to test the sample, etc.

In some embodiments, providing a test sample comprises isolating a nucleic acid molecule or a polynucleotide, where the nucleic acid molecule or the polynucleotide is separated from other nucleic acid molecules or polynucleotides which are present in the natural source of the nucleic acid molecule or polynucleotide.

The term “isolated” in the context of nucleic acid molecules or polynucleotides refers to a nucleic acid molecule or polynucleotide which is separated from other nucleic acid molecules or polynucleotides which are present in the natural source of the nucleic acid molecule or polynucleotide. Moreover, an “isolated” nucleic acid molecule or polynucleotide, such as a cDNA molecule, can be substantially free of other cellular material or culture medium when produced by recombinant techniques, or substantially free of chemical precursors or other chemicals when chemically synthesized. In one aspect, nucleic acid molecules or polynucleotides are isolated.

In some embodiments, isolating a test sample comprises extracting and storing nucleic acids from a sample, comprising the steps of providing the sample onto a solid matrix comprising a protein denaturant and acid or acid-titrated buffer reagent, generating an acidic pH for extraction of the nucleic acids from the sample upon hydration of the solid matrix with the sample or any externally added liquid, drying the matrix comprising extracted nucleic acids, and storing the extracted nucleic acids on the matrix in a substantially dry state under ambient temperature. The isolation may further comprise a step of providing a sample, applying a sample or disposing a sample on the extraction matrix using a pipet, catheter, syringe or conduit. The sample may be poured on the matrix.

Aurora Kinase Inhibitor

The term “Aurora kinase inhibitor” or “inhibitor of Aurora kinase” is used to signify a compound which is capable of interacting with an Aurora kinase and inhibiting its enzymatic activity. Inhibiting Aurora kinase enzymatic activity means reducing the ability of an Aurora kinase to phosphorylate a substrate peptide or protein. In various embodiments, such reduction of Aurora kinase activity is at least about 50%, at least about 75%, at least about 90%, at least about 95%, or at least about 99%. In various embodiments, the concentration of Aurora kinase inhibitor required to reduce an Aurora kinase enzymatic activity is less than about 1 μM, less than about 500 nM, less than about 100 nM, or less than about 50 nM.

In some embodiments, the Aurora kinase inhibitor for treating the eligible patient according to the present invention is selected from group consisting of Aurora kinase A inhibitors, Aurora kinase B inhibitors, Aurora kinase C inhibitors, and the mixtures thereof.

Preferably, the Aurora kinase for the eligible patient is an “Aurora kinase B inhibitor.” The term “Aurora kinase B inhibitor” refers to a therapeutic compound of any type (e.g., non-selective or selective), including small molecule-, antibody-, antisense-, small interfering RNA, or microRNA-based compounds, that binds to at least one of Aurora kinase B or Aurora B, and antagonizes the activity of the Aurora kinase B or Aurora B related nucleic acid or protein. For example, a number of Aurora kinase B inhibitors are known to inhibit at least one of histone H3 phosphorylation or cell division. In addition, a number of Aurora kinase B inhibitors are known to induce apoptosis in at least one cell system (such as an acute myeloid leukemia cell line, a primary acute myeloid leukemia culture, etc.). The methods of the present invention are useful with any known or hereafter developed Aurora kinase B inhibitor. Examples of Aurora kinase B inhibitors include, but are not limited to AZD1152, ZM447439, VX-680/MK0457, ABT-348 and Hesperadin.

AZD1152, also known as, 2-[[3-({4-[(5-{2-[(3-Fluorophenyl)amino]-2-oxoethyl}-1H-pyrazol-3-yl)amino]-quinazolin-7-yl}oxy)propylliethyl)amino]ethyl dihydrogen phosphate, is a prodrug of a pyrazoloquinazoline Aurora kinase inhibitor (AZD1152-hydroxyquinazolien pyrazol anilide (HQPA)) and is converted rapidly to the active AZD1152-HQPA in plasma (See, Mortlock, A A, et al., J. Med. Chem., 50:2213-24 (2007)). AZD1152-HQPA is a highly potent and selective inhibitor of Aurora B.

ZM447439, also known as 4-(4-(N-benzoylamino)anilino)-6-methoxy-7-(3-(1-morpholino)propoxy)quinazoline, is a quinazoline derivative and inhibits Aurora A and Aurora B. The chemical structure of ZM447439 is provided in Ditchfield, C., et al., J. Cell Bio., 161(2):267-280 (2003) and Montembault, E., et al., Drugs of the Future, 30(1):1-9 (2005).

VX-680/MK0457 is a cyclopropane carboxylic acid of {4-[4-(4-methyl-piperazin-1-yl)-6-(5-methyl-2H-pyrazol-3-ylamino)-pyrimidin-2-ylsulphanyl]-phenyl}-amide and inhibits Aurora A, Aurora B and Aurora C. The chemical structure of VX-680/MK0457 is provided in Montembault, E., et al., Drugs of the Future, 30(1):1-9 (2005).

Hesperadin, an indolinone, inhibits Aurora B. The chemical structure of Hesperadin is provided in Hauf, S., et al., J. Cell Bio., 161(2):281-294 (2003) and Montembault, E., et al., Drugs of the Future, 30(1):1-9 (2005).

In the most preferred embodiment, the “Aurora kinase B inhibitor” of the present invention is 1-(4-(4-amino-7-(1-(2-hydroxyethyl)-1H-pyrazol-4-yl)thieno[3,2-c]pyridin-3-yl)phenyl)-3-(3-fluorophenyl)urea (ABT-348).

Treatment

The terms “treat”, “treating” or “treatment” as used herein refer to administering one or more active agents or compounds to a subject in an effort to (i) prevent a pathologic condition from occurring (e.g. prophylaxis); (ii) inhibit the pathologic condition or arrest its development; (iii) relieve a pathologic condition and/or prevent or reduce the severity one or more symptoms associated with such a pathologic condition, regardless of whether any of items (i) through (iii) are successful in a subject.

In some embodiments, the treatment of the present invention is to administer a therapeutic composition comprising at least one Aurora kinase inhibitor selected from a group consisting of Aurora kinase A inhibitors, Aurora kinase B inhibitors, Aurora kinase C inhibitors, and mixtures thereof.

Preferably, the therapeutic composition for the treatment according to the present invention comprises an Aurora kinase B inhibitor selected from the group consisting of AZD1152, ZM447439, VX-680/MK0457, 1-(4-(4-amino-7-(1-(2-hydroxyethyl)-1H-pyrazol-4-yl)thieno[3,2-c]pyridin-3-yl)phenyl)-3-(3-fluorophenyl)urea (ABT-348), and Hersperadin.

More preferably, the Aurora kinase B inhibitor for the treatment is 1-(4-(4-amino-7-(1-(2-hydroxyethyl)-1H-pyrazol-4-yl)thieno[3,2-c]pyridin-3-yl)phenyl)-3-(3-fluorophenyl)urea (ABT-348).

In some embodiments, the treatment is for cancer.

The term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

In another embodiment of the methods herein, the treatment is for cancer, and the cancer is selected from the group consisting of acoustic neuroma, acute leukemia, acute lymphoblastic leukemia, acute myelogenous leukemia, acute t-cell leukemia, basal cell carcinoma, bile duct carcinoma, bladder cancer, brain cancer, breast cancer, bronchogenic carcinoma, Burkitt's lymphoma, cervical cancer, chondrosarcoma, chordoma, choriocarcinoma, chronic leukemia, chronic lymphocytic leukemia, chronic myelocytic leukemia, chronic myelogenous leukemia, colon cancer, colorectal cancer, craniopharyngioma, cystadenocarcinoma, dysplasias, metaplasias, embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, gastric carcinoma, germ cell testicular cancer, gestational trophobalstic disease, glioblastoma, head and neck cancer, heavy chain disease, hemangioblastoma, hepatoma, hepatocellular cancer, hormone insensitive prostate cancer, leiomyosarcoma, liposarcoma, lung cancer, lymphangioendothelio-sarcoma, lymphangiosarcoma, lymphoblastic leukemia, lymphoma, malignancies and hyperproliferative disorders of the bladder, breast, colon, lung, ovaries, pancreas, prostate, skin and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, oligodendroglioma, oral cancer, osteogenic sarcoma, ovarian cancer, pancreatic cancer, papillary adenocarcinomas, papillary carcinoma, peripheral T-cell lymphoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors, stomach cancer, squamous cell carcinoma, synovioma, sweat gland carcinoma, testicular cancer, thyroid cancer, Waldenstrom's macroglobulinemia, testicular tumors, uterine cancer, and Wilms' tumor. In another embodiment of the methods herein, the treatment is for cancer, and the cancer is acute myeloid leukemia.

The treatment can also further comprise one or more other types of treatment, for example, but not limited to chemotherapy, radiation or combinations thereof.

In one embodiment, the patient is also being treated with chemotherapy.

In another embodiment, the patient is also being treated with radiation.

In another embodiment, the patient is also being treated with chemotherapy and radiation.

### Polynucleotide Assays

A preferred nucleic acid assay method useful in the invention comprise detection of the presence or absence of copy number increases or decreases by: (i) in situ hybridization assays to intact tissue or cellular samples, (ii) microarray hybridization assays to chromosomal DNA extracted from a tissue sample, and (iii) polymerase chain reaction (PCR) or other amplification assays to chromosomal DNA extracted from a tissue sample. Assays using synthetic analogs of nucleic acids, such as peptide nucleic acids, in any of these formats can also be used.

In preferred embodiments, the methods of the invention are used to identify copy number increases or decreases for CDKN2A locus for use in both classification of patients and for monitoring patient response to Aurora kinase B inhibitor therapy. Assays for classification can be run before start of therapy, and patients that do not show or exhibit showing a copy number decrease CDKN2A are eligible to receive Aurora kinase B inhibitor therapy. For monitoring patient response, the assays can be run at the initiation of therapy to establish baseline levels of the CDKN2A in the tissue sample, for example, the percent of total cells or number of cells showing the copy number in the sample. The same tissue is then sampled and assayed and the levels of the CDKN2A compared to the baseline. Where the levels remain the same or decrease, the therapy is likely being effective and can be continued. Where significant increase over baseline level occurs, the patient may not be responding or may have developed resistance to continued Aurora kinase B inhibitor therapy.

The assays of the invention can be used with targeted cancer therapy, such as targeted therapies to solid tumors (e.g., sarcomas or carcinomas) or hematological malignancies (e.g., cancers that affect blood, bone marrow, and lymph nodes). The assays of the present invention can be used with solid tumors such as colorectal carcinoma, pancreatic carcinoma, thyroid cancer, prostate cancer, bladder cancer, liver cancer, bile duct cancer, oral cancer, non-small-cell lung carcinoma, small-cell lung carcinoma, ovarian cancer or breast cancer. The assays of the present invention can be used with hematological malignancies such as acute lymphocytic leukemia (ALL), acute myeloid leukemia (AML), chronic myelogenous leukemia (CML), diffuse large B-cell lymphoma (DLBCL), Philadelphia chromosome-positive acute lymphoblastic leukemia (Ph+ALL) and chronic lymphocytic leukemia (CLL). The assays can be performed in relation to any cancer type in which amplification or over-expression of Aurora kinase is involved. The inventive assays are performed on any type of test sample, such as a patient tissue sample of any type or on a derivative thereof, including peripheral blood, tumor or suspected tumor tissues (including fresh frozen and fixed paraffin-embedded tissue), cell isolates such as circulating epithelial cells separated or identified in a blood sample, lymph node tissue, bone marrow and fine-needle aspirates.

The present invention comprises detection of the genomic biomarker CDKN2A by hybridization assays using detectably labeled nucleic acid-based probes, such as deoxyribonucleic acid (DNA) probes or protein nucleic acid (PNA) probes, or unlabeled primers which are designed/selected to hybridize to a specific chromosomal target. The unlabeled primers are used in amplification assays, such as by polymerase chain reaction (PCR), in which after primer binding, a polymerase amplifies the target nucleic acid sequence for subsequent detection. The detection probes used in PCR or other amplification assays are preferably fluorescent, and still more preferably, detection probes useful in “real-time PCR”. Fluorescent labels are also preferred for use in situ hybridization but other detectable labels commonly used in hybridization techniques, e.g., enzymatic, chromogenic and isotopic labels, can also be used. Useful probe labeling techniques are described in the literature (Fan, Y.-S. 2002. Molecular cytogenetics: protocols and applications. Humana Press, Totowa, N. J. xiv, p. 411, the contents of which are incorporated herein by reference). In detection of the genomic biomarkers by microarray analysis, these probe labeling techniques are applied to label a chromosomal DNA extracted from a patient sample, which is then hybridized to the microarray.

Preferably, in situ hybridization is used to detect the presence of chromosomal copy number increase for CDKN2A. Primer and probes can be made by one of skill in the art using the sequences of CDKN2A (SEQ ID NO:1). In some embodiments, the CDKN2A may comprise p16 gene, so the primers and probes can be made using the sequence of p16 gene (SEQ ID NO:2).

Probes for use in the in situ hybridization methods of the invention fall into two broad groups: chromosome enumeration probes, i.e., probes that hybridize to a chromosomal region, usually a repeat sequence region, and indicate the presence or absence of an entire chromosome; and locus specific probes, i.e., probes that hybridize to a specific locus on a chromosome and detect the presence or absence of a specific locus. Chromosome arm probes, i.e., probes that hybridize to a chromosomal region and indicate the presence or absence of an arm of a specific chromosome, can also be used. It is preferred to use a locus specific probe that can detect changes of the unique chromosomal DNA sequences at the interrogated locus, such as the ABCB1 and ABCB4 loci. Methods for use of unique sequence probes for in situ hybridization are described in U.S. Pat. No. 5,447,841, the contents of which are incorporated herein by reference.

A chromosome enumeration probe can hybridize to a repetitive sequence, located either near or removed from a centromere, or can hybridize to a unique sequence located at any position on a chromosome. For example, a chromosome enumeration probe can hybridize with repetitive DNA associated with the centromere of a chromosome.

One of exemplary probes used in the present invention is CDKN2A and CEPS FISH probe mix which is commercially available from Abbott Molecular Diagnostics (Catalog No. 04N61-020). The probe comprises a ⁻222kb sequence which maps at the position Chr9:21788971-22011133 of Human GRCh37/hg19 assembly:

For example, the probe comprises a sequence of SEQ ID NO: 3 or a sequence substantially identical to SEQ ID NO:3.

Probe
SEQ ID NO: 3
GTGCTGTCACTCCCTTTACCAGTGGGTCTGCATTTTCCTTTGCACTACAA
GTACTCTAACTACAGACATTGTTCCGGTTTTTATCTGATTATAAGTATCC
AACTAAAGTAGTTAAATGTTTAGAAAAGGTAAATAACTGATAGTGCATAA
GAGAACCTGAAGTACTCTGTTGACACTTAAATTATATAGCCAAGGACAAT
GGAGGAAATCATTTAGTGCTATTAATCATTAATCTAACAGTATTGTGTCT
GTGTGCAACTGTCTTTCTTTTGGCTAATATCTGTGAATTAATTGGTCATT
GAAAAATCTGATAAAGGAATATTCTAAAAGTACACAGTGAAATCCAAATT
ATTTCTATCAATTACAGCTCACCTTTTCTGAGGCTTAAACTTGACATGTA
AAAACATTTTAGTTATCTGTTTCAAAATGAACAATATACCTCTGGTTAAA
ATTCTACCTTTTAAAAATTAAGGTATTTGAGACCTACAGGGATTAATTCT
CTACTTAATATTTACTTTACTTTAGTAGTTTTTCTACAGAAAGTTTGTGA
AAGATTTCATCCCCTATATATGTTTCTTTATCCTCCAAAATCTGGTCCTA
CCACTGTACTCCTTGA

The probe may also comprise a sequence of SEQ ID NO:4 or a sequence substantially identical to SEQ ID NO: 4.

Probe
SEQ ID NO: 4
CCTGAGTATAAGCAAAGCGCTGTCCGTGGTGCCCCTTTAAAGGGAATGCC
CCCGGCAACGTTTTCCCTACCCAAAGCTTTTATTCATTCAGTCAACTTGC
TTCGCGAAGCTCACACATCTGCCTCGTGCAAGATTCTCAGTCATTTTACT
TAGTCATTGGTTCTTTCCCTATCACCATTCTTTATGTCCCCCTCAAAGAA
AAACATTATCTTCCATTTCCTTATCAACTCCAAACAGCTTTCATTTTTCT
GACATATTTACTACCTAAGAAAATGGCTCAAGAATTGGGTCAGACTATCT
TGTCCTAACTTTTCTGATAAGTTTCAGAGAAACTCAAAGGTCAAAACAAG
AGCATAAGAGTAAAAGGTAGAGTTTTTTTTAAACTGAAGACTAGGAAATG
GGGGTTGGATGGGAAAGAAAAAGAAATTGTTATTAATGCTACCCGGTTCC
CTTCCCTGTCCAGGTGGATTTCAGCTCTGTTGAGGCTCTGTCAGTAGATT
TTCAGCCCTAACCAGCACTTCCATGGTGGTGGCACTTCCACTGCCTTTAA
AAGAAAGAGCTTTTTTTAATTCTACAGGGATTTGGGGGATGAGGAGTCAG
AGCTAAGGTATCCTAAAAAAAACATGTGAAGACTCTCATTTTGCAATACA
CAAGCAATTGCCCTCCTGTTAAGACTTTGTCTTCCTCAGCACTCCGAACC
AAAATGATTCTGTAAACAAAAATTGTTCACTTTTAGGAGAGGTCCACTTA
TGCAGTTCCTCACCCAAGTTTTTAGGCAACAAATCCATAACTTGCGGTTC
TCTTCCTATCCAATGTAGCATCCGCTGAAATGTTTTAAATATTTTAAGTA
ATAA

Exceptionally useful in situ hybridization probes are directly labeled fluorescent probes, such as described in U.S. Pat. No. 5,491,224, incorporated herein by reference. U.S. Pat. No. 5,491,224 also describes simultaneous FISH assays using more than one fluorescently labeled probe.

Useful locus specific probes can be produced in any manner and generally contain sequences to hybridize to a chromosomal DNA target sequence of about 10,000 to about 1,000,000 bases long. Preferably the probe hybridizes to a target stretch of chromosomal DNA at the target locus of at least 100,000 bases long to about 500,000 bases long and also includes unlabeled blocking nucleic acid in the probe mix, as disclosed in U.S. Pat. No. 5,756,696, the contents of which are herein incorporated by reference, to avoid non-specific binding of the probe. It is also possible to use unlabeled, synthesized oligomeric nucleic acid or peptide nucleic acid as the blocking nucleic acid. For targeting the particular gene locus, it is preferred that the probes include nucleic acid sequences that span the gene and thus hybridize to both sides of the entire genomic coding locus of the gene. The probes can be produced starting with human DNA-containing clones such as Bacterial Artificial Chromosomes (BAC's) or the like. BAC libraries for the human genome are available from Invitrogen (Carlsbad, Calif.) and can be investigated for identification of useful clones. It is preferred to use the University of California Santa Cruz Genome Browser to identify DNA sequences in the target locus. These DNA sequences can then be used to synthesize PCR primers for use to screen BAC libraries to identify useful clones. The clones can then be labeled by conventional nick translation methods and tested as in situ hybridization probes.

Examples of fluorophores that can be used in the in situ hybridization methods described herein are: 7-amino-4-methylcoumarin-3-acetic acid (AMCA); Texas Red™ (Molecular Probes, Inc., Eugene, Oreg.); 5-(and-6)-carboxy-X-rhodamine; lissamine rhodamine B; 5-(and-6)-carboxyfluorescein; fluorescein-5-isothiocyanate (FITC); 7-diethylaminocoumarin-3-carboxylic acid, tetramethyl-rhodamine-5-(and-6)-isothiocyanate; 5-(and-6)-carboxytetramethylrhodamine; 7-hydroxy-coumarin-3-carboxylic acid; 6-[fluorescein 5-(and-6)-carboxamido]hexanoic acid; N-(4,4-difluoro-5,7-dimethyl-4-bora-3a, 4a diaza-3-indacenepropionic acid; eosin-5-isothiocyanate; erythrosine-5-isothiocyanate; 5-(and-6)-carboxyrhodamine 6G; and CascadeTM blue aectylazide (Molecular Probes; an Invitrogen brand).

Probes can be viewed with a fluorescence microscope and an appropriate filter for each fluorophore, or by using dual or triple band-pass filter sets to observe multiple fluorophores. See, for example, U.S. Pat. No. 5,776,688, the contents of which are incorporated herein by reference. Any suitable microscopic imaging method can be used to visualize the hybridized probes, including automated digital imaging systems. Alternatively, techniques such as flow cytometry can be used to examine the hybridization pattern of the chromosomal probes.

Although the cell-by-cell gene amplification analysis resulting from in situ hybridization is preferred, the genomic biomarkers can also be detected by quantitative PCR. In this embodiment, chromosomal DNA is extracted from the tissue sample, and is then amplified by PCR using a pair of primers specific to CDKN2A, or by multiplex PCR, using multiple pairs of primers. Any primer sequence for the biomarkers can be used.

Microarray-based copy number analysis can also be used. In one embodiment, the chromosomal DNA after extraction is labeled for hybridization to a microarray comprising a substrate having multiple immobilized unlabeled nucleic acid probes arrayed at probe densities up to several million probes per square centimeter of substrate surface. Multiple microarray formats exist and any of these can be used, in the present invention. Examples of microarrays that can be used are the Affymetrix GeneChip® Mapping 100K Set SNP Array (See Matsuzaki, H., et al., “Genotyping over 100,000 SNPs on a pair of oligonucleotide arrays,” Nat Methods. 1:109-11 (2004)); the Affymetrix GeneChip® Mapping 250K Assay Kits (such as the GeneChip® Human Mapping 250K Nsp Array or the GeneChip® Human Mapping 250K Sty Array) or the Affymetrix GeneChip® Mapping 500K Array Set, each of which is commercially available from Affymetrix, Inc., Santa Clara, Calif.), the Agilent Human Genome aCGH Microarray 44B (available from Agilent Technologies, Inc., Santa Clara, Calif.), Illumina microarrays (Illumina, Inc., San Diego, Calif.), Nimblegen aCGH microarrays (Nimblegen, Inc., Madison, Wis.), etc. When using an oligonucleotide microarray to detect amplifications, it is preferred to use a microarray that has probe sequences to more than three separate locations in the targeted region.

Detecting Expression: mRNA

The level of gene expression for the CDKN2A can be determined by assessing the amount of one or more mRNAs in the test sample. Methods of measuring mRNA in samples are known in the art. To measure mRNA levels, the cells in a test sample can be lysed, and the levels of mRNA in the lysates or in RNA purified or semi-purified from lysates can be measured by any variety of methods familiar to those in the art. Such methods include hybridization assays using detectably labeled DNA or RNA probes (i.e., Northern blotting) or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled (e.g., fluorescent, or enzyme-labeled) DNA or RNA probes. Additional methods for quantifying mRNA include RNA protection assay (RPA), cDNA and oligonucleotide microarrays, representation difference analysis (RDA), differential display, EST sequence analysis, and serial analysis of gene expression (SAGE).

In suitable embodiments, PCR amplification is used to detect for the CDKN2A in the test sample. Briefly, in PCR, two primer sequences are prepared that are complementary to regions on opposite complementary strands of the marker sequence, for example containing the sequences for the CDKN2A. An excess of deoxynucleotide triphosphates are added to a reaction mixture along with a DNA polymerase, e.g., Taq polymerase. If the target sequence is present in a sample, the primers will bind to the sequence and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated, thereby generating amplification products. A reverse transcriptase PCR amplification procedure can be performed in order to quantify the amount of mRNA amplified.

Any suitable fragment of the CDKN2A can be amplified and detected. Designing efficient primers for PCR is within the ordinary skill in the art. Examples of primers that can be used are shown in Table 3. Typically, amplified fragments for detection are approximately 50 to 300 nucleotides in length.

Amplification products can be detected in several ways. Amplification products can be visualized by electrophoresis of the sample in a gel and then staining with a DNA binding dye, e.g., ethidium bromide. Alternatively, the amplification products can be integrally labeled with a radio- or fluorescence nucleotide and then visualized using x-ray film or under the appropriate stimulating spectra.

Amplification can be also monitored using “real-time” methods. Real-time PCR allows for the detection and quantitation of a nucleic acid target. Typically, this approach to quantitative PCR utilizes a fluorescent dye, which can be a double-strand specific dye, such as SYBR GREEN®. Alternatively, other fluorescent dyes (e.g., FAM or HEX) can be conjugated to an oligonucleotide probe or a primer. Various instruments capable of performing real time PCR are known in the art and include, for example, the ABI PRISM® 7900 (Applied Biosystems) and LIGHTCYCLER® systems (Roche). The fluorescent signal generated at each cycle of PCR is proportional to the amount of PCR product. A plot of fluorescence versus cycle number is used to describe the kinetics of amplification and a fluorescence threshold level is used to define a fractional cycle number related to initial template concentration. When amplification is performed and detected on an instrument capable of reading fluorescence during thermal cycling, the intended PCR product from non-specific PCR products can be differentiated using melting analysis. By measuring the change in fluorescence while gradually increasing the temperature of the reaction subsequent to amplification and signal generation it can be possible to determine the Tm of the intended product(s) as well as that of the nonspecific product.

The methods can include amplifying multiple nucleic acids in sample, also known as “multiplex detection” or “multiplexing.” Multiplex PCR″ refers to PCR that involves adding more than one set of PCR primers to the reaction in order to detect and quantify multiple nucleic acids, including nucleic acids from one or more target gene markers. Furthermore, multiplexing with an internal control (e.g., 18S rRNA, GADPH, or actin) provides a control for the PCR without reaction.

The methods can also be based on a signal amplification assay, also known as “branched DNA assay.” Several different short single-stranded DNA molecules can be used in a branched DNA-assay. For example, the capture and capture-extender oligonucleotide bind to the target nucleic acid and immobilize it on a solid support. The branched DNA binds to the sample nucleic acid by specific hybridization in areas which are not occupied by capture hybrids. The label oligonucleotide and the branched DNA then detect the immobilized target nucleic acid.

### Sample Processing and Assay Performance

As discussed previously herein, the test sample of the present invention can be a tissue sample. The tissue sample to be assayed by the methods of the present invention can comprise any type, including a peripheral blood sample, a tumor tissue or a suspected tumor tissue, a thin layer cytological sample, a fine-needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, and this extract can then be assayed. A microdissection of the tissue sample to obtain a cellular sample enriched with suspected tumor cells can also be used. The preferred tissue samples for use herein are peripheral blood, tumor tissue or suspected tumor tissue, including fine needle aspirates, fresh frozen tissue and paraffin embedded tissue, and bone marrow.

The tissue sample can be processed by any desirable method for performing in situ hybridization or other nucleic acid assays. For the preferred in situ hybridization assays, a paraffin embedded tumor tissue sample or bone marrow sample is fixed on a glass microscope slide and deparaffinized with a solvent, typically xylene. Useful protocols for tissue deparaffinization and in situ hybridization are available from Abbott Molecular Inc. (Des Plaines, Ill.). Any suitable instrumentation or automation can be used in the performance of the inventive assays. PCR based assays can be performed on the m2000 instrument system (Abbott Molecular, Des Plaines, Ill.). Automated imaging can be used for the preferred fluorescent in situ hybridization assays.

In one embodiment, the sample comprises a peripheral blood sample from a patient which is processed to produce an extract of circulating tumor or cancer cells to be examined for the presence or absence of a copy number increase or decrease for the CDKN2A. The circulating tumor cells can be separated by immunomagnetic separation technology such as that available from Immunicon (Huntingdon Valley, Pennsylvania). The copy number determined for the circulating tumor cells is then compared to the baseline level or predetermined level of circulating tumor cells having a copy number determined at a previous point in time, such as at the start of therapy. Increases in the copy number compared to the baseline level or the predetermined level can indicate therapy failure.

Test samples can comprise any number of cells that is sufficient for a clinical diagnosis, and typically contain at least about 100 cells. In a typical FISH assay, the hybridization pattern is assessed in about 25-1,000 cells. Test samples are typically considered “test positive” when found to contain the gene amplification in a sufficient proportion of the sample. The number of cells identified with chromosomal copy number and used to classify a particular sample as positive, in general, varies with the number of cells in the sample. The number of cells used for a positive classification is also known as the cut-off value. Examples of cut-off values that can be used in the determinations include about 5, 25, 50, 100 and 250 cells, or 5%, 10%, 15%, 20%, 25%, 30%, 35%, 40%, 50% and 60% of cells in the sample population. As little as one cell can be sufficient to classify a sample as positive. In a typical paraffin embedded tissue sample, it is preferred to identify at least 30 cells as positive and more preferred to identify at least 20 cells as positive for having the chromosomal copy number gain. For example, detection in a typical paraffin embedded colorectal carcinoma of 30 cells would be sufficient to classify the tissue as positive and eligible for treatment.

### Kits

The present invention also contemplates kits for detecting the presence or absence of a copy number gain the CDKN2A in a test sample. Such kits can comprise one or more reagents for determining the presence or absence of the above described copy number gain. For example, said kit can contain one or more nucleic acid probes. Alternatively, or in addition to the probes, the kit can contain one or more nucleic acid primers.

Thus, the present disclosure further provides for diagnostic and quality control kits comprising one or more nucleic acid primers, nucleic acid probes or nucleic acid primers and probes described herein. Optionally the assays, kits and kit components of the present invention can be optimized for use on commercial platforms (e.g., immunoassays on the Prism®, AxSYM®, ARCHITECT® and EIA (Bead) platforms of Abbott Laboratories, Abbott Park, Ill., as well as other commercial and/or in vitro diagnostic assays). Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories, Abbott Park, Ill.) electrochemical immunoassay system that performs sandwich immunoassays for several cardiac markers, including TnI, CKMB and BNP. Immunosensors and methods of operating them in single-use test devices are described, for example, in U.S. Patent Application Publication Nos. 2003/0170881, 2004/0018577, 2005/0054078 and 2006/0160164, which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (e.g., sensitivity panels, calibrators, and positive controls). Preparation of quality control reagents is well known in the art, and is described, e.g., on a variety of immunodiagnostic product insert sheets.

The kit can incorporate a detectable label, such as a fluorophore, radioactive moiety, enzyme, biotin/avidin label, chromophore, chemiluminescent label, or the like, or the kit may include reagents for labeling the nucleic acid primers, the nucleic acid probes or the nucleic acid primers and nucleic acid probes for detecting the presence or absence of a copy number gain as described herein. The primers and/or probes, calibrators and/or controls can be provided in separate containers or pre-dispensed into an appropriate assay format, for example, into microtiter plates.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit further can optionally include instructions for use, which may be provided in paper form or in computer-readable form, such as a disc, CD, DVD or the like.

The kit (or components thereof), as well as the method of determining the presence or absence of a copy number gain for the CDKN2A using the components and methods described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., capture antibody) is attached (which can impact sandwich formation and analyte reactivity), and the length and timing of the capture, detection and/or any optional wash steps.

Whereas a non-automated format such as an ELISA may require a relatively longer incubation time with sample and capture reagent (e.g., about 2 hours) an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, and U.S. Patent. Application Publication Nos. 2003/0170881, 2004/0018577, 2005/0054078, and 2006/0160164, which are incorporated in their entireties by reference for their teachings regarding same.

It further goes without saying that the methods and kits as described herein necessarily encompass other reagents and methods for carrying out the assays. For instance, encompassed are various buffers such as those known in the art and/or which can be readily prepared or optimized to be employed.

### EXAMPLES

### Reagents

Antibodies were purchased from the indicated suppliers as follows: rabbit monoclonal anti-p16 (Catalog No. 3562-1) were from Epitomics (Burlingame, Calif.), anti-actin antibody (Catalog No. A5441) was from Sigma. (MO), secondary anti-rabbit 680 was from Invitrogen (Carlsbad, Calif.). All Aurora kinase inhibitors and ABT-869 (144-(3-amino-1H-indazol-4-yl)phenyl]-3-(2-fluoro-5-methylphenyl)urea, Linifanib) were synthesized (AbbVie).

The chemical structures of MLN8237 (4-{[9-Chloro-7-(2-fluoro-6-methoxyphenyl)-5H-pyrimido[5,4-d][2]benzazepin-2-yl]amino}-2-methoxybenzoic acid, Alisertib) inhibits Aurora A), AZD1152 (2-[[3-({4-[(5-{2-[(3-Fluorophenyl)amino]-2-oxoethyl}-1H-pyrazol-3-yl)amino]-quinazolin-7-yl)oxy)propylliethyl)amino]ethyl Dihydrogen Phosphate), and VX-680/MK-0457 (cyclopropane carboxylic acid {4-[4-(4-methyl-piperazin-1-yl)-6-(5-methyl-2H-pyrazol-3-ylamino)-pyrimidin-2-ylsulphanyl]-phenyl}-amide), AT9283 (1-Cyclopropyl-3-(3-(5-(morpholinomethyl)-1H-benzo[d]imidazol-2-yl)-1H-pyrazol-4-yl)urea), ABT-869 and ABT-348 have been disclosed and are readily available in the literature.

Cell Culture and Generation of Stable p16 shRNA-OCI-AML2 Cell Line

AML cell lines were obtained from the American Type Culture Collection (ATCC; Manassas, Va.) and propagated according to ATCC recommendations.

OCI AML2 cells were infected with control or three p16 shRNA from Sigma (MO). Infected cells were selected with 1 μM puromycin. After selection, cells were used in drug treatment experiments. All cells were maintained at 37° C. in 5% CO₂.

### Flow Cytometry

Blood of patients was added to lyse fix buffer (BD Biosciences). After lysis of red blood cells, the sample was washed, and permeabilized using Phosflow Perm/wash buffer (BD Biosciences). The sample was incubated for 15 minutes with DAPI (75 ng/mL) before analyzing on a flow cytometer.

### Fish

CDKN2A and CEPS FISH probe mix was obtained from Abbott Molecular Diagnostics (Catalog No. 04N61-020).

### Results

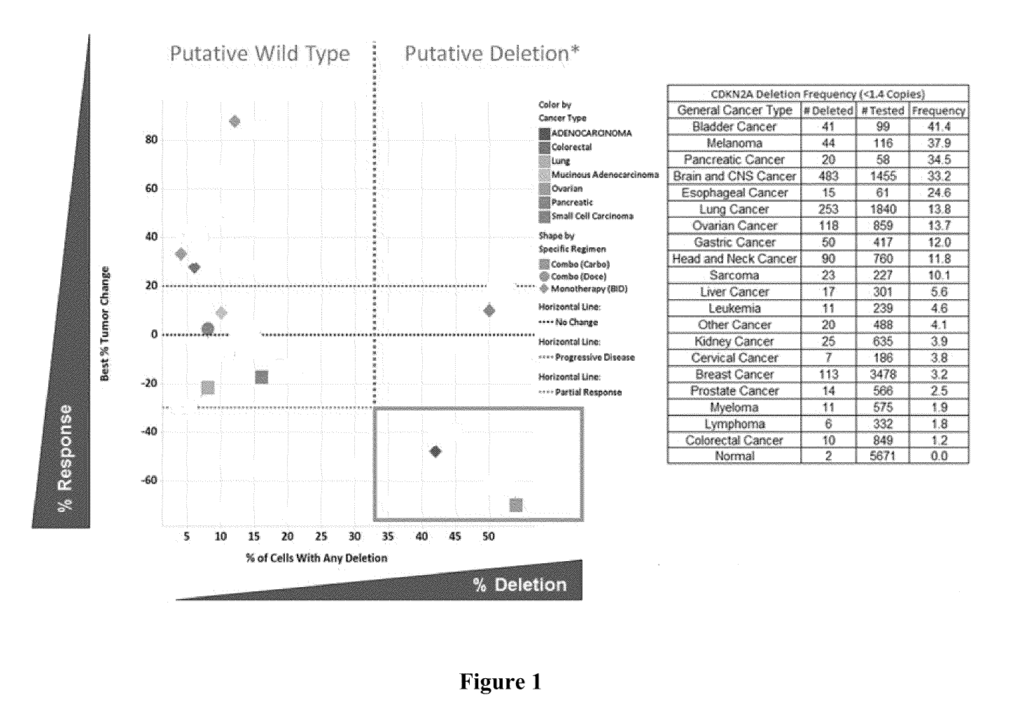

As shown in FIG. 1 (left panel), deletion associated with clinical response in patients with solid tumors treated with ABT-348 is found in clinical trial. The Y axis list best % tumor change from baseline and the X axis are % tumor cells assessed with a deletion in CDKN2A. The hashed line indicates a putative cutoff separating patients with deletion from those without. Only three patients showed a deletion of CDKN2A. Two of these patients had a clinical response and the third discontinued due to toxicity and was unevaluable for tumor changes. No patient without a deletion demonstrated a tumor response. The frequency of CDKN2A deletion across a wide range of tumor types is shown in FIG. 1 (right panel). Array CGH data from compendia bioscience database Oncomine used. Threshold to define deletion is <1.2 copies.

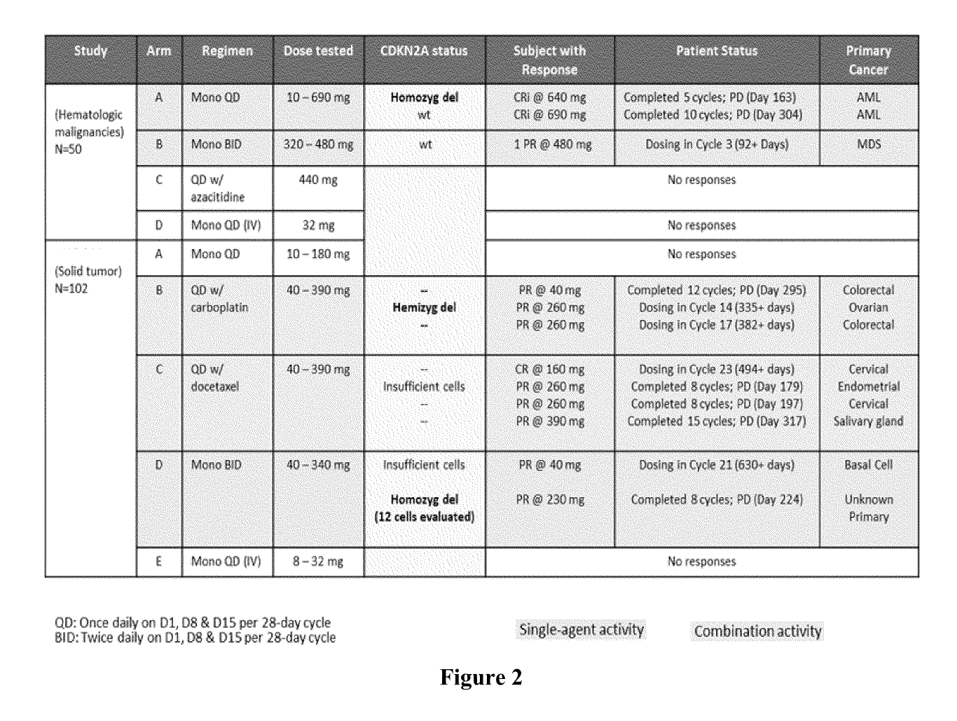

FIG. 2 shows CDKN2A status of Objective Responders in Phase 1 clinical trial of ABT-348 in subjects with hematologic malignancies. Three patients assessed had deletion of CDKN2A. One patient obtained a complete response, and the others had a partial response.

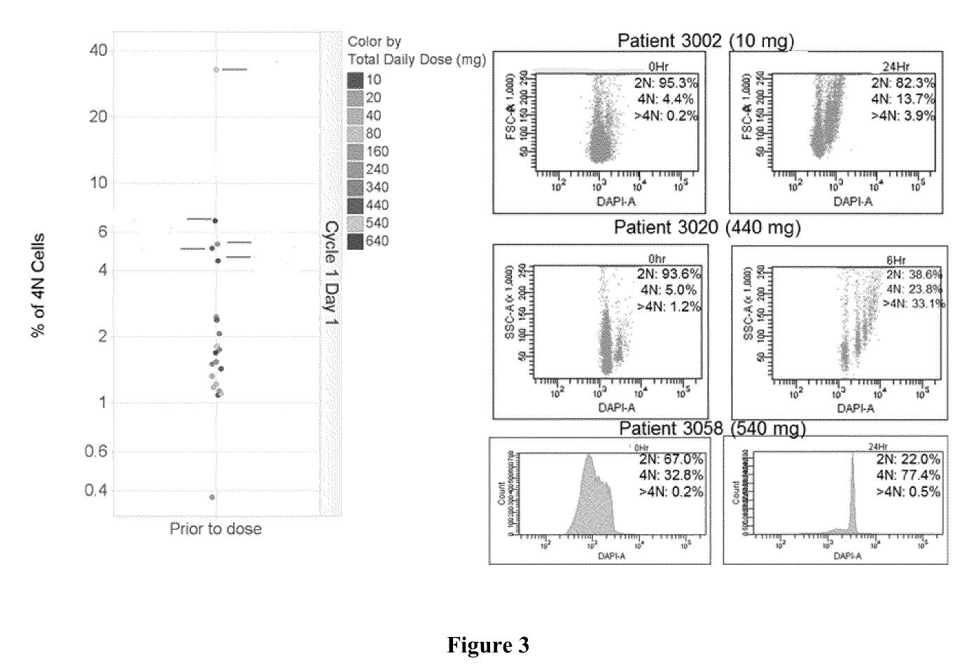

FIG. 3 shows percentage of cycling cells at baseline associated with pharmacodynamic activity of patients treated with ABT-348. The left panel shows the percentage of cycling cells prior to dose. The right panel shows examples of pharmacodynamics activity (increase in the number of ≧4N cells).

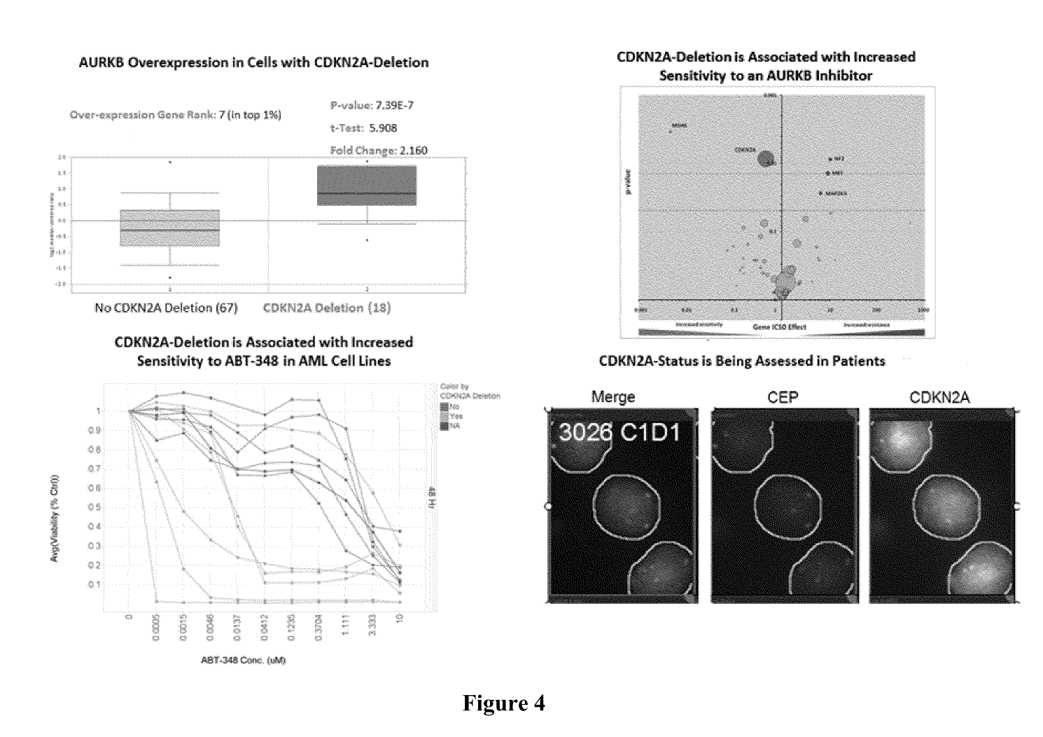

FIG. 4 shows the identification of ABT-348 as a sensitivity marker. Based on observation, it was hypothesized that patient samples with a high proportion of cells in G2/M (ie. cycling) would be sensitive to ABT-348. Therefore, a database search was performed to identify genotypes likely to overexpress Aurora kinase B given its expression is increased in M phase of cell cycle (top left). CDKN2A deletion was found to have significantly higher Aurora kinase B expression, therefore cells harboring this deletion are predicted to be more sensitive to Aurora kinase inhibitors. Data from Cancer Cell Line Encyclopedia (CCLE) supported this hypothesis (top right). Sensitivity to Aurora kinase inhibitors including ABT-348 was assessed in a panel of cell lines with and without this deletion (bottom left). The data showed cell lines with CDKN2A deletion were more sensitive to all Aurora kinase inhibitors tested (data for ABT-348 are shown). CDKN2A status was then assessed by in situ hybridization in patient samples from ABT-348 trials. An example from one patient is shown (bottom right).

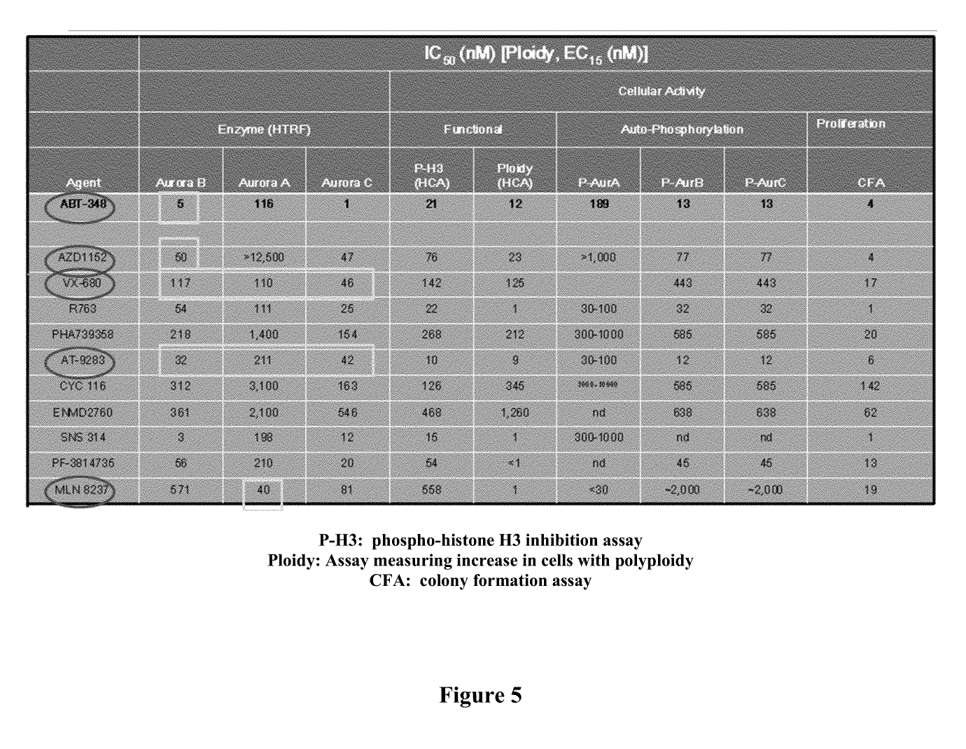

FIG. 5 shows relative potencies of other Aurora kinase inhibitors. The circled inhibitors were tested in cell line panel.

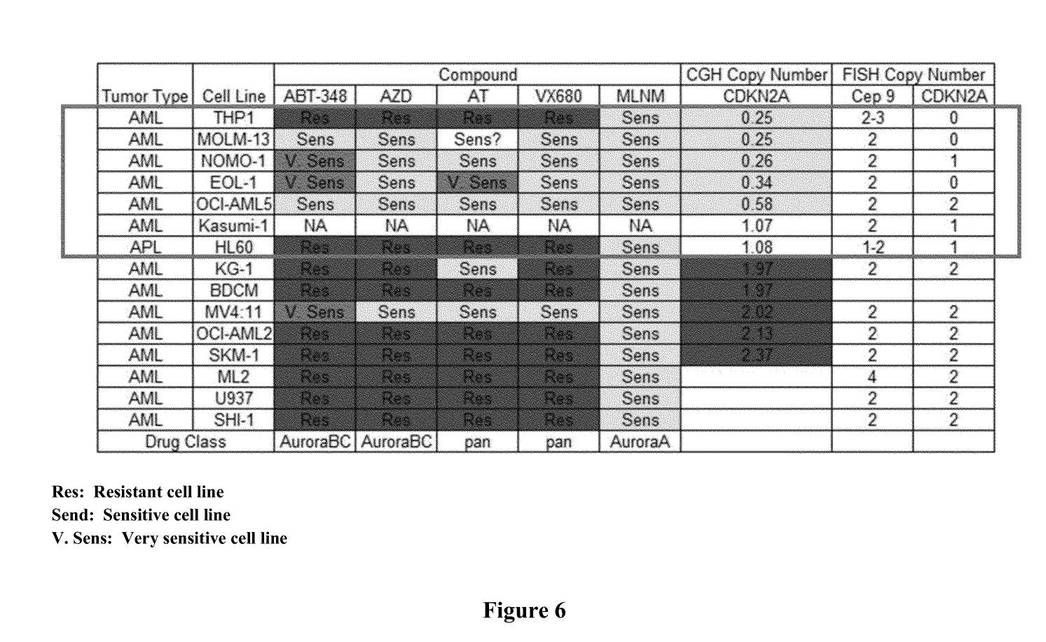

FIG. 6 shows that the predictive nature of CDKN2A extends to multiple Aurora kinase inhibitors.

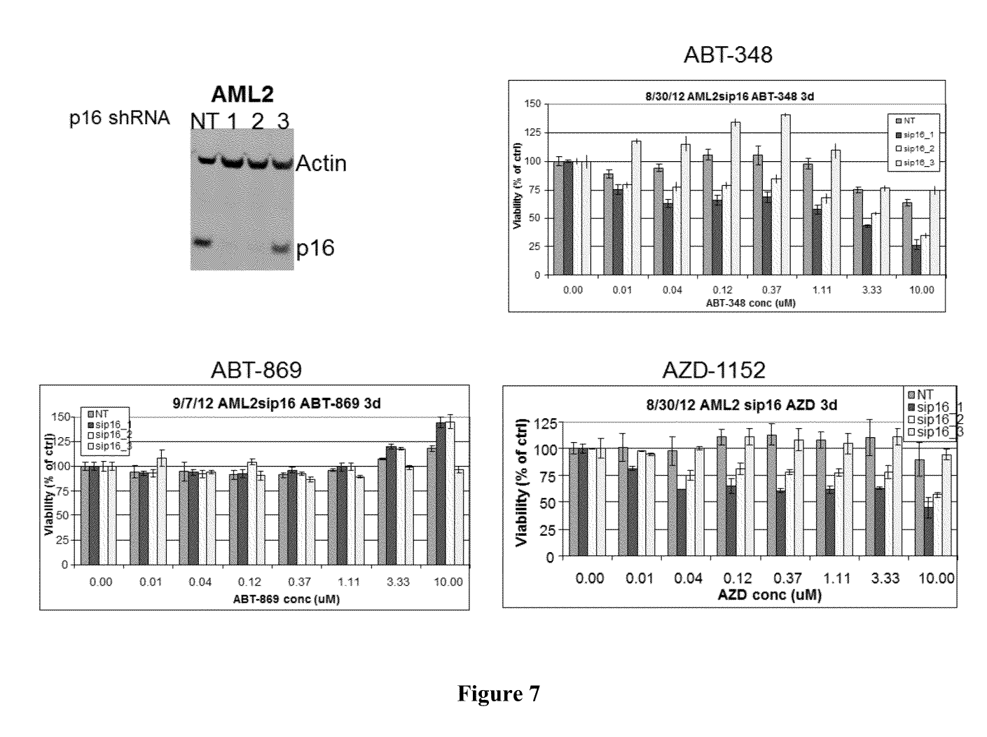

FIG. 7 shows that silencing p16 sensitizes OCI-AML2 cells to Aurora kinase B inhibitor compounds but not ABT-869. One gene product of the CDKN2A locus is p16. Therefore shRNA was used to decrease p16 expression to mimic a situation of CDKN2A deletion. The p16 knockdown made cells more sensitive to Aurora kinase B inhibitors ABT-348 and AZD-1152. Given ABT-348 is a dual inhibitor of VEGF and Aurora kinase B inhibitors, ABT-869, which inhibits VEGF but not Aurora kinase B inhibitors was included to confirm the effect was specific to Aurora kinase B inhibitors inhibition.

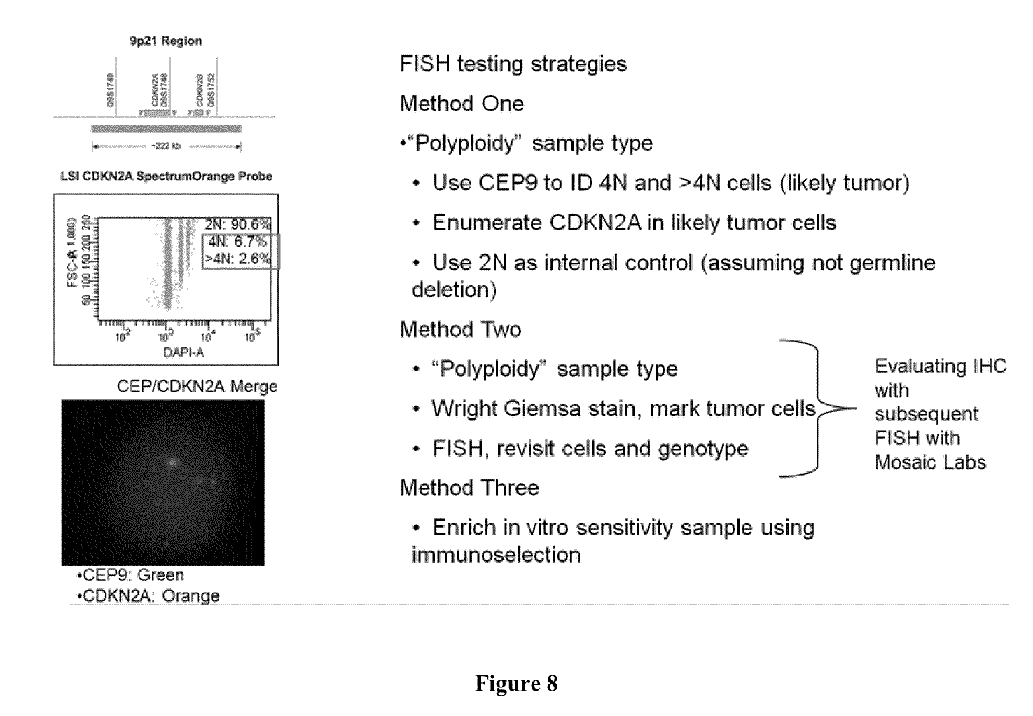

FIG. 8 shows the approaches that were employed to test CDKN2A status clinically.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method of classifying a patient for eligibility for treatment with an Aurora kinase inhibitor, the method comprising the steps of:
a) providing a test sample from a patient;
b) determining the presence or absence of a copy number decrease of the cyclin dependent kinase 2A (CDKN2A) locus in the sample; and
c) classifying the patient as being eligible for receiving treatment with an Aurora kinase inhibitor if the copy number decrease of the CDKN2A locus is present.

2. The method of claim 1, wherein the CDKN2A locus comprises SEQ ID NO:1 or a sequence substantially identical to SEQ ID NO:1.

3. The method of claim 1, wherein the CDKN2A comprises a p16 gene.

4. The method of claim 3, wherein the p16 gene comprises SEQ ID NO:2 or a sequence substantially identical to SEQ ID NO:2.

5. The method of claim 1, wherein the determining step (b) is performed by in situ hybridization.

6. The method of claim 5, wherein the determining step (b) is performed with at least one probe comprising a sequence complementary to CDKN2A.

7. The method of claim 6, wherein the probe comprises a sequence comprising a sequence complementary to SEQ ID NO:1.

8. The method of claim 5, wherein the determine step (b) is performed with at least one probe comprising a sequence complementary to p16.

9. The method of claim 8, wherein the probe comprises a sequence complementary to SEQ ID NO:2.

10. The method of claim 6 or 9, wherein the probe comprises at least one sequence selected from group consisting of SEQ ID NO:3 and SEQ ID NO:4.

11. The method of claim 6 or 9, wherein the probe is fluorescently labeled.

12. The method of claim 5, wherein the determining step (b) is performed by polymerase chain reaction.

13. The method of claim 5, wherein the determining step (b) is performed by a nucleic acid microarray assay.

14. The method of claim 1, wherein the Aurora kinase inhibitor is selected from the group consisting of Aurora kinase A inhibitors, Aurora kinase B inhibitors, Aurora kinase C inhibitors, and mixtures thereof.

15. The method of claim 14, wherein the Aurora kinase inhibitor is 1-(4-(4-amino-7-(1-(2-hydroxyethyl)-1H-pyrazol-4-yl)thieno[3,2-c]pyridin-3-yl)phenyl)-3-(3-fluorophenyl)urea.

16. The method of claim 1, wherein the patient is also being treated with chemotherapy, radiation or combinations thereof.

17. The method of claim 1, wherein the test sample comprises a tissue sample.

18. The method of claim 17, wherein the tissue sample comprises a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a tumor tissue or a suspected tumor tissue, a thin layer cytological sample, a fine needle aspirate sample, a bone marrow sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample.

19. A method of monitoring a patient suffering from cancer and being treated with an Aurora kinase inhibitor, the method comprising the steps of:
a) providing a test sample from a patient suffering from cancer and currently being treated with at least one Aurora kinase inhibitor;
b) determining a copy number for the cyclin dependent kinase 2A (CDKN2A) locus in the sample;
c) comparing the copy number of the CDKN2A locus in the sample against a predetermined level; and
d) determining that the patient should continue to be treated with the Aurora kinase inhibitor if the copy number of the CDKN2A locus in the sample is lower than the predetermined level.

20. The method of claim 19, wherein the CDKN2A comprises a p16 gene.

21. A kit for classifying a patient for eligibility for treatment with an Aurora kinase inhibitor comprising:
(a) at least one reagent capable of detecting the presence or absence of a copy number decrease of the cyclin dependent kinase 2A (CDKN2A) locus in the sample, wherein the presence of the copy number decrease of the CDKN2A locus indicates the patient as being eligible for receiving treatment with an Aurora kinase inhibitor.

22. The kit of claim 21, wherein the CDKN2A comprises a p16 gene.

23. The kit of claim 21, wherein the reagent comprises at least one probe comprising a sequence complementary to the CDKN2A.

24. The kit of claim 21, wherein the reagent comprises at least one probe comprising a sequence complementary to the p16 gene.

25. The kit of claim 21, wherein the reagent comprises at least one antibody immunologically specified for the proteins encoded by CDKN2A.

26. The kit of claim 25, wherein the antibody is used in an ELISA.

27. A kit for monitoring a patient suffering from cancer and being treated with an Aurora kinase inhibitor comprising:
(a) at least one reagent capable of detecting the presence or absence of a copy number decrease of the cyclin dependent kinase 2A (CDKN2A) locus in the sample, wherein the presence of the copy number decrease of the CDKN2A locus indicates that the patient should continue to be treated with the Aurora kinase inhibitor.

28. The kit of claim 27, wherein the CDKN2A comprises a p16 gene.

29. The kit of claim 27, wherein the reagent comprises at least one probe comprising a sequence complementary to the CDKN2A.

30. The kit of claim 27, wherein the reagent comprises at least one probe comprising a sequence complementary to the p16 gene.

31. The kit of claim 27, wherein the reagent comprises at least one antibody immunologically specified for the proteins encoded by CDKN2A.

32. The kit of claim 31, wherein the antibody is used in an ELISA.
